(19) United States
(12) Patent Application Publication
(10) Pub. No.: US 2009/0036440 A1
(43) Pub. Date: Feb. 5, 2009

(54) NOVEL PYRIMIDINE DERIVATIVES - 816

(75) Inventors: Bernard Christophe BARLAAM, Reims (FR); Richard DUCRAY, Reims (FR)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 12/109,618

(22) Filed: Apr. 25, 2008

(30) Foreign Application Priority Data
Apr. 27, 2007 (EP) 07301001.9
Apr. 27, 2007 (EP) 07301002.7

(51) Int. Cl. A61K 31/5377; C07D 413/14; A61P 35/00

(52) U.S. Cl. 514/232.2; 544/122; 514/235.8; 544/82

(57) ABSTRACT

The invention concerns pyrimidine compounds of Formula I, or a pharmaceutically acceptable salt thereof,

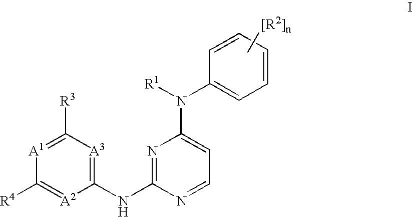

where A¹, A² A³, R¹, n, R², R³, and R⁴ are as defined in the description. The present invention also relates to processes for the preparation of such compounds, pharmaceutical compositions containing them and their use in the manufacture of a medicament for use as an antiproliferative agent in the prevention or treatment of tumours or other proliferative conditions which are sensitive to the inhibition of EphB4 kinases.

This application claims the benefit under 35 U.S.C. § 119(a)-(d) of Application No. 07301001.9 (EP) filed on 27 Apr. 2007 and Application No. 07301002.7 (EP) filed on 27 Apr. 2007.

The present invention relates to novel pyrimidine derivatives, to pharmaceutical compositions containing these derivatives and to their use in therapy, in particular in the prevention and treatment of cancer, in a warm blooded animal such as man.

Many of the current treatment regimes for cell proliferation diseases such as psoriasis and cancer utilise compounds which inhibit DNA synthesis. Such compounds are generally toxic to all cells, but their toxic effects on rapidly dividing cells, such as tumour cells, can be beneficial.

In recent years it has been discovered that a cell may become cancerous by virtue of the transformation of a portion of its DNA into an oncogene i.e. a gene which, on activation, leads to the formation of malignant tumour cells (Bradshaw, Mutagenesis, 1986, 1, 91). Several such oncogenes give rise to the production of peptides which are receptors for growth factors. Activation of the growth factor receptor results in an increase in cell proliferation. It is known, for example, that several oncogenes encode tyrosine kinase enzymes and that certain growth factor receptors are also tyrosine kinase enzymes (Yarden et al, Ann. Rev. Biochem. 1988, 57, 443; Larsen et al., Ann. Reports in Med. Chem. 1989, Chpt. 13).

Receptor tyrosine kinases play an important role in the transmission of biochemical signals, which initiate a variety of cell responses—including cell proliferation, survival and migration. They are large enzymes which span the cell membrane and possess an extracellular binding domain for growth factors, such as epidermal growth factor (EGF), and an intracellular portion which functions as a kinase to phosphorylate tyrosine amino acids in proteins and thereby influence cell proliferation. A large number of receptor tyrosine kinases are known (Wilks, Advances in Cancer Research, 1993, 60 43-73) and are classified on the basis of the family of growth factors that bind to the extracellular domain. This classification includes Class I receptor tyrosine kinases comprising the EGF family of receptor tyrosine kinases such as the EGF, TGFα, Neu and erbB receptors, Class II receptor tyrosine kinases comprising the insulin family of receptor tyrosine kinases such as the insulin and IGF1 receptors and insulin-related receptor (IRR), and Class III receptor tyrosine kinases comprising the platelet-derived growth factor (PDGF) family of receptor tyrosine kinases such as the PDGFα, PDGFβ and colony-stimulating factor 1 (CSF1) receptors.

The Eph family is the largest known family of receptor tyrosine kinases, with 14 receptors and 8 cognate ephrin ligands identified in mammals (reviewed in Kullander and Klein, Nature Reviews Molecular Cell Biology, 2002, 3, 475-486). The receptor family is further sub-divided into two sub-families defined largely by homology of extracellular domains and affinity towards a particular ligand type. In general, all Eph receptors contain an intracellular tyrosine kinase domain and an extracellular Ig-like domain with a cysteine-rich region with 19 conserved cysteines and two fibronectin type III domains. The A-class of Eph receptors consists of 8 receptors termed EphA1-8, which generally bind to their cognate ephrinA class of ligands termed ephrinA1-5. The B-class consists of 6 receptors termed EphB1-6, which bind to their cognate ephrinB ligands termed ephrinB1-3. Eph receptor ligands are unusual and differ to most other receptor tyrosine kinase ligands in that they are also tethered to cells, via a glycosylphosphatidylinositol linker in ephrinA ligands or an integral transmembrane region in ephrinB ligands. The binding of ephrin ligand to the Eph receptor induces a conformational change within the Eph intracellular domain that enables phosphorylation of tyrosine residues within an auto-inhibitory juxtamembrane region, which relieves this inhibition of catalytic site and enables additional phosphorylation to stabilise the active conformation and generate more docking sites for downstream signalling effectors.

Furthermore, evidence indicates that Eph/ephrin signalling can regulate other cell responses, such as proliferation and survival.

There is growing evidence that Eph receptor signalling may contribute to tumourigenesis in a wide variety of human cancers, either on tumour cells directly or indirectly via modulation of vascularisation. For instance, many Eph receptors are over-expressed in various tumour types (Reviewed in Surawska et al., Cytokine & Growth Factor Reviews, 2004, 15, 419-433, Nakamoto and Bergemann, Microscopy Res and Technique, 2002, 59, 58-67). The expression of EphB receptors, including EphB4, is up-regulated in tumours such as neuroblastomas, leukemias, breast, liver, lung and colon. Furthermore, various in vitro and in vivo studies particularly relating to EphB4 have indicated that over-expression of Eph receptors on cancer cells is able to confer tumourigenic phenotypes such as proliferation and invasion, consistent with the speculated role in oncogenesis.

For instance, inhibition of EphB4 expression using interfering-RNA or antisense oligodeoxynucleotides inhibited proliferation, survival and invasion of PC3 prostate cancer cells in vitro and in vivo xenograft model (Xia et al., Cancer Res., 2005, 65, 4623-4632.

In addition to compelling role of Eph receptors on tumour cells, there is good evidence that EphB4 may contribute to tumour vascularisation (Reviewed in Brantley-Sieders et al., Current Pharmaceutical Design, 2004, 10, 3431-3442, Cheng et al., Cytokine and Growth Factor Reviews, 2002, 13, 75-85). Members of Eph family including EphB4 are expressed on endothelial cells. Transgenic studies have shown that disruption of EphB4 (Gerety et al., Molecular Cell, 1999, 4, 403-414) or its ligand ephrinB2 (Wang et al., Cell, 1998, 93, 741-753) causes embryonic lethality associated with vascular modelling defects consistent with a critical role in vessel development. EphB4 activation stimulates endothelial cell proliferation and migration in vitro (Steinle et al., J. Biol. Chem., 2002, 277, 43830-43835).

Moreover, inhibition of EphB4 signalling using soluble extracellular-domains of EphB4 have been shown to inhibit tumour growth and angiogenesis in in vivo xenograft studies (Martiny-Baron et al., Neoplasia, 2004, 6, 248-257, Kertesz et al., Blood, 2005, Pre-published online).

Accordingly it has been recognised that an inhibitor of Eph receptors, particularly EphB4, should be of value as a selective inhibitor of the proliferation and survival of tumour cells by either targeting the tumour cells directly or via their effects on tumour vascularisation. Thus, such inhibitors should be valuable therapeutic agents for the containment and/or treatment of tumour disease.

The applicants have found that certain pyrimidine compounds are useful in the inhibition of EphB4 and therefore may be useful in therapy for the treatment of disease states in which increased EphB4 activity is implicated.

According to a first aspect of the invention, there is provided a compound of Formula I

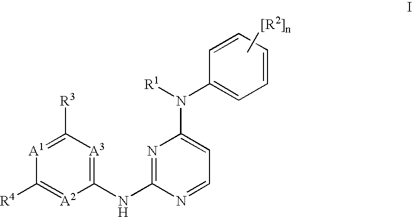

wherein:
one of A¹, A² or A³ is N, and the others are independently selected from CH or N;
R¹ is a (1-4C)alkyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, —NR^{a}CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R⁸ is hydrogen or (1-2C)alkyl;
R³ is selected from:
(i) hydrogen, halo, nitro, cyano, or hydroxy;
(ii) an optionally substituted (1-6C)alkyl, (2-6C)alkenyl, or (2-6C)alkynyl group wherein the optional substituents are selected from: cyano; halo; a group of sub-formula:

—W—R⁹
wherein W is selected from —O—, —S(O)ₚ— (where p is 0, 1 or 2), —CO—, —NR^{b}CO—, —CONR^{b}—, —NR^{b}CONR^{b}—, —SO₂NR^{b}—, —NR^{b}SO₂—, or —NR^{b}COO—;
R^{b} is selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen, or (1-2C)alkyl, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iii) a group —NR¹²R¹³, wherein R¹² and R¹³ are each independently selected from hydrogen or (1-6C)alkyl, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iv) a group of formula (II):

-X-R¹⁴
wherein X is selected from —O—, —S(O)ₚ— (where p is 0, 1 or 2), —CO—, —NR^{c}CO—, —CONR^{c}—, —NR^{c}COO—, and —NR^{c}SO₂—,
where R^{c} is selected hydrogen or (1-2C)alkyl;
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl; or
(v) a 4-7 membered heterocyclic group which is linked via a carbon atom;
   R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
   or a pharmaceutically acceptable salt thereof.

It is to be understood that, insofar as certain of the compounds of Formula I defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by the resolution of a racemic form. Similarly, the above-mentioned activity may be evaluated using the standard laboratory techniques referred to hereinafter.

It is to be understood that certain compounds of Formula I defined above may exhibit the phenomenon of tautomerism. In particular, tautomerism may affect any heterocyclic groups that bear 1 or 2 oxo substituents. It is also to be understood that the present invention includes in its definition any such tautomeric form, or a mixture thereof, which possesses the above-mentioned activity and is not to be limited merely to any one tautomeric form utilised within the Formulae drawings or named in the Examples.

It is to be understood that certain compounds of Formula I above may exist in unsolvated forms as well as solvated forms, such as, for example, hydrated forms. It is also to be understood that the present invention encompasses all such solvated forms that possess anticancer or antitumour activity.

It is also to be understood that certain compounds of the Formula I may exhibit polymorphism, and that the present invention encompasses all such forms which possess anticancer or antitumour activity.

It is further to be understood that any R² group that is present on the phenyl moiety of the aniline group that is located at the 4-position on the pyrimidine ring may be located at any available position on said phenyl moiety. When multiple R² groups are present, each R² group may be the same or different.

In this specification the generic term “alkyl” includes both straight-chain and branched-chain alkyl groups such as propyl, isopropyl and tert-butyl. However references to individual alkyl groups such as “propyl” are specific for the straight-chain version only, references to individual branched-chain alkyl groups such as “isopropyl” are specific for the branched-chain version only. An analogous convention applies to other generic terms, for example (1-4C)alkoxy includes methoxy, ethoxy and isopropoxy.

The term “halo” refers to fluoro, chloro, bromo, or iodo.

The term “heterocyclic ring” or “heterocyclic group” unless otherwise defined herein, refers to saturated, partially saturated or unsaturated monocyclic rings containing 4, 5, 6 or 7 ring atoms wherein at least one of said atoms, and suitably from 1-4 or said atoms, is a heteroatom, such as oxygen, sulphur or nitrogen. Where they are unsaturated, they may be aromatic, and such rings are described as “heteroaryl” groups.

In particular compounds of the invention, “heterocyclic rings” are saturated monocyclic rings that contain 4, 5, 6 or 7 ring atoms, and especially 5 or 6 ring atoms.

Examples and suitable values of the term “heterocyclic ring” used herein are pyrrolidinyl, imidazolidinyl, pyrazolidinyl, piperidinyl, piperazinyl, morpholin-4-yl, thiomorpholin-4-yl, 1,4-oxazepan-4-yl, diazepanyl and oxazolidinyl.

Examples of “heteroaryl” rings include thienyl, furanyl, pyrrolyl, imidazolyl, pyrazolyl, thiazolyl, isothiazolyl, isoxazolyl, oxazolyl, thiadiazolyl, oxadiazolyl, triazolyl, tetrazolyl, pyridinyl, pyrazinyl, pyridazinyl or pyrimidinyl.

Particular novel compounds of the invention include, for example, compounds of Formula I, or pharmaceutically-acceptable salts thereof, wherein, unless otherwise stated, each of R¹, n, R², R³ or R⁴ has any of the meanings defined hereinbefore or in paragraphs (1) to (40) hereinafter:—
(1) R¹ is (1-4C)alkyl;
(2) R¹ is selected from methyl, ethyl, propyl, isopropyl, 2-methylpropyl or cyclopropylmethyl;
(3) R¹ is selected from methyl, ethyl, isopropyl or cyclopropylmethyl;
(4) R¹ is methyl;
(5) R¹ is isopropyl;
(6) R¹ is cyclopropylmethyl;
(7) R¹ is ethyl;
(8) n is 1, 2 or 3;
(9) n is 2 or 3;
(10) n is 1;
(11) n is 2;
(12) n is 3;
(13) each R² group present is independently selected from (1-2C)alkyl, (1-2C)alkoxy, fluoro, chloro, cyano, hydroxy(1-2C)alkyl, or a group of sub-formula:

-Q-R⁸
where Q is selected from —NR^{a}—CO—, —S(O)_{z}— (where z is 0, 1 or 2); R^{a} is selected from hydrogen or methyl, and R⁸ is hydrogen or (1-2C)alkyl;
(14) each R² group present is independently selected from (1-2C)alkyl, (1-2C)alkoxy, fluoro, chloro, cyano, hydroxy(1-2C)alkyl, or a group of sub-formula:

-Q-R⁸
where Q is selected from —NR^{a}—CO—, —S(O)_{z}— (where z is 0, 1 or 2); R^{a} is selected from hydrogen or methyl, and R⁸ is hydrogen or (1-2C)alkyl;
(15) each R² group present is independently selected from methyl, fluoro, chloro, hydroxymethyl, methoxy, acetamido, or methylthio;
(16) each R² group present is independently selected from methyl, fluoro, chloro, hydroxymethyl, or methoxy;
(17) each R² group present is independently selected from fluoro or chloro;
(18) each R² group present is independently selected from methyl or hydroxymethyl;
(19) each R² group present is methyl;
(20) each R² group present in hydroxymethyl;
(21) each R² group present is independently selected from acetamido or methoxy;
(22) each R² group present is methoxy;
(23) R³ is selected from:
   (i) hydrogen, halo, nitro, cyano, or hydroxy;
   (ii) an optionally substituted (1-6C)alkyl group, wherein the optional substituents are selected from cyano, halo,
   or a group of sub-formula:

—W—R⁹
wherein W is selected from —O—, —S(O)ₚ— (where p is 0, 1 or 2), —CO—, —NR^{b}CO—, or —CONR^{b}—; R^{b} is selected from hydrogen or (1-2C)alkyl;
and R⁹ is selected from hydrogen or (1-4C)alkyl;
or —NR¹⁰R¹¹ where R¹⁰ and R¹¹ are independently selected from hydrogen, (1-2C)alkanoyl or (1-2C)alkyl, or R¹⁰ and R¹¹ are linked to form a 5, or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
(iii) a group —NR¹²R¹³, wherein R¹² and R¹³ are each independently selected from hydrogen or (1-6C)alkyl, or R¹² and R¹³ are linked to form a 5, 6 or 7-membered heterocyclic ring which comprises, in addition to the nitrogen atom to which R¹² and R¹³ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl; or
(iv) a group of formula (II):

-X-R¹⁴
wherein X is selected from —O—, —S(O)ₚ— (where p is 0, 1 or 2), —CO—, NR^{c}CO—, CONR^{c}, or NR^{c}COO—,
where R^{c} is selected hydrogen or (1-2C)alkyl;
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 5, or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
(24) R³ is selected from:
   (i) hydrogen, halo, cyano, or hydroxy;
   (ii) an optionally substituted (1-4C)alkyl group wherein the optional substituents are selected from cyano, halo,
   a group of sub-formula:

—W—R⁹
wherein W is selected from —O—, —S(O)ₚ— (where p is 0, 1 or 2), —CO—, —NR^{b}CO—, or —CONR^{b}—; R^{b} is selected from hydrogen or (1-2C)alkyl
and R⁹ is selected from hydrogen or (1-4C)alkyl;
or —NR¹⁰R¹¹, where R¹⁰ and R¹¹ are independently selected from hydrogen or (1-2C)alkyl, or R¹⁰ and R¹¹ are linked to form a 5 or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl;
(iii) a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl; or
(iv) a group of formula (II):

-X-R¹⁴
wherein X is selected from —O—, —S(O)ₚ— (where p is 0, 1 or 2), or —CONR^{c}—,
where R^{c} is selected hydrogen or (1-2C)alkyl;
R¹⁴ is a (1-4C)alkyl group which is optionally substituted by halo, hydroxy, cyano, (1-4C)alkoxy;
(25) R³ is selected from:
   (i) hydrogen, halo, or cyano;
   (ii) an optionally substituted (1-2C)alkyl group wherein the optional substituents are selected from cyano, halo, a group of sub-formula:

—W—R⁹
wherein W is selected from —O—, —S(O)ₚ— (where p is 0, 1 or 2), —CO—, —NR^{b}CO—, or —CONR^{b}—; R¹ is selected from hydrogen or (1-2C)alkyl
and R⁹ is selected from hydrogen or (1-4C)alkyl;
or —NR¹⁰R¹¹, where R¹⁰ and R¹¹ are independently selected from hydrogen or (1-2C)alkyl), or R¹⁰ and R¹¹ are linked to form a 5 or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl;
(iii) a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl; or
(iv) a group of formula (TI):

-X-R¹⁴
wherein X is selected from —O—, —S(O)ₚ— (where p is 0, 1 or 2), or —CONR^{c}—,
where R^{c} is selected hydrogen or (1-2C)alkyl;
R¹⁴ is a (1-4C)alkyl group which is optionally substituted by halo, hydroxy, cyano, (1-4C)alkoxy;
(26) R³ is a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
(27) R³ is halo such as chloro;
(28) R³ is a 4-7 membered heterocyclic group which is linked via a carbon atom;
(29) R³ is a 5-6 membered heterocyclic group which is linked via a carbon atom;
(30) R³ is a 5-6 membered heteroaryl group which is linked via a carbon atom;
(31) R³ is selected from carbon linked pyrrolidinyl, imidazolidinyl, pyrazolidinyl, piperidinyl, piperazinyl, morpholinyl, thiomorpholinyl, oxazolidinyl, thienyl, furanyl, pyrrolyl, imidazolyl, pyrazolyl, thiazolyl, isothiazolyl, isoxazolyl, oxazolyl, oxadiazolyl, thiadiazolyl, triazolyl, tetrazolyl, pyridinyl, pyrazinyl, pyridazinyl or pyrimidinyl;
(31.1) R³ is selected from a nitrogen linked piperidinyl, piperazinyl or morpholinyl ring, wherein any carbon atom present in the ring is optionally substituted by hydroxy and the available nitrogen atom present in the piperazinyl ring is optionally substituted by methyl;
(32) R⁴ is a group —NR¹⁷R¹⁸, wherein R¹⁷ and R¹⁸ are linked to form a 5 or 6 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl, hydroxy(1-4C)alkyl, or (1-4C)alkanoyl;
(33) R⁴ is a group —NR¹⁷R¹⁸, wherein R¹⁷ and R¹⁸ are linked to form a 6 membered heterocyclic ring which optionally comprises, in addition to the nitrogen atom to which R¹⁷ and R¹⁸ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom is optionally substituted by (1-4C)alkyl, hydroxy(1-4C)alkyl or (1-4C)alkanoyl;
(34) R⁴ is a group of Formula:

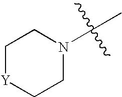

wherein Y is selected from O, S, NR^{y}, or CR^{z}, where R^{y} is selected from hydrogen, (1-2C)alkyl, hydroxy(1-2C)alkyl, (1-2C)alkoxy(1-2C)alkyl, or (1-2C)alkanoyl, and R^{z} is selected from hydrogen, hydroxy, (1-2C)alkyl, hydroxy(1-2C)alkyl, (1-2C)alkoxy(1-2C)alkyl, or (1-2C)alkanoyl;
(35) R⁴ is a group of Formula:

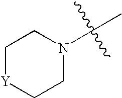

wherein Y is selected from O, NR^{y}, or CR^{z}, where R^{y} is selected from hydrogen or (1-2C)alkyl, and R^{z} is selected from hydrogen or hydroxy;
(36) R⁴ is selected from morpholin-4-yl, 4-methylpiperazin-1-yl, or 4-hydroxypiperidin-1-yl;
(37) R⁴ is morpholin-4-yl;
(38) A¹ or A² is nitrogen;
(39) A² or A³ is CH;
(40) A¹ is nitrogen and A² and A³ are CH.

In a particular group of compounds of the Formula I, R¹ is an alkyl group as defined in any one of paragraphs (1) to (7) above. In a further group of compounds of the invention, R¹ is methyl.

In a further group of compounds of Formula I, n is an integer selected from 0, 1, 2 or 3, and each R² group that may be present is as defined in any one of paragraphs (13) to (22) above. In a particular group of compounds of the invention, each R² group present is as defined in any one of paragraphs (15) to (22) above.

In a further sub-group of compounds of the invention, n is 3 and each R² group is selected from fluoro or chloro.

In a further group of compounds, the aniline in the 4-position of the pyrimidine ring has the following structure:

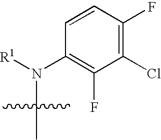

wherein R¹ has any one of the definitions set out herein.

In a further sub-group of compounds, n is 2 and each R² group present is selected from methyl or hydroxymethyl.

In a further group of compounds, the aniline in the 4-position of the pyrimidine ring has the following structure:

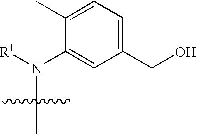

wherein R¹ has any one of the definitions set out herein.

In a further sub-group of compounds, n is 1 and the R² group present is methoxy.

In a further group of compounds, the aniline in the 4-position of the pyrimidine ring has the following structure:

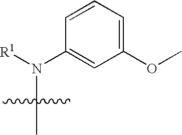

wherein R¹ has any one of the definitions set out herein.

In a further particular group of compounds of the invention, R³ is as defined in any one of paragraphs (23) to (27) above, and is especially as defined in paragraphs (25), (26) or (27) above.

In a particular group of compounds of the invention, R⁴ is as defined in any one of paragraphs (32) to (37) above. In a further particular group of compounds of the invention, R⁴ is as defined in either paragraph (36) or (37). Suitably, R⁴ is morpholin-4-yl.

In a group of compounds of Formula I, n, R¹ and R³ have any one of the definitions set out hereinbefore, R⁴ is a group of Formula:

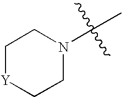

wherein Y is —NR^{y}—, and R^{y} is selected from hydrogen or (1-2C)alkyl, and each R² group present is independently selected from (1-2C)alkyl, (1-2C)alkoxy, fluoro, chloro, cyano, hydroxy(1-2C)alkyl, or a group of sub-Formula:

-Q-R⁸

where Q is selected from —CO—, —NR^{a}—, or —S(O)_{z}— (where z is 0, 1 or 2); R^{a} is selected from hydrogen or methyl, and R⁸ is hydrogen or (1-2C)alkyl.

In a further group of compounds of Formula I, R⁴ is a group of Formula:

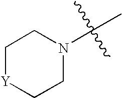

wherein Y is —NR^{y}—, and R^{y} is selected from hydrogen or (1-2C)alkyl, and each R² group present is independently selected from (1-2C)alkyl, (1-2C)alkoxy, fluoro, chloro, cyano, or hydroxy(1-2C)alkyl.

In a sub-group of compounds of Formula I, R⁴ is a group of Formula:

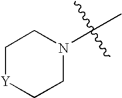

wherein Y is O or —CR^{z}—, and R^{z} is selected from hydrogen or hydroxy, and R¹, R², n and R³ each have any one of the definitions set out hereinbefore.

In a particular sub-group of compounds, A¹, A² and/or A³ are as defined in paragraphs (38)-(40) above. Suitably one or two of A¹, A² or A³ is nitrogen, and in particular one of A¹, A² or A³ is nitrogen whilst the other are CH. In particular, there are nitrogen atoms at the A¹ and/or A² position. In a particular embodiment, A¹ is nitrogen and A² and A³ are CH. In an alternative embodiment A² is nitrogen and A¹ and A³ are CH.

A particular sub-group of compounds of the invention, or pharmaceutically acceptable salts thereof, have the structural Formula IA:

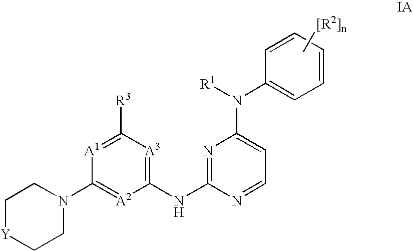

wherein:
Y is selected from O, S, NR^{y}, or CR^{z}, where R^{y} is selected from hydrogen, (1-2C)alkyl, hydroxy(1-2C)alkyl, (1-2C)alkoxy(1-2C)alkyl, or (1-2C)alkanoyl, and R^{z} is selected from hydrogen, hydroxy, (1-2C)alkyl, hydroxy(1-2C)alkyl, (1-2C)alkoxy(1-2C)alkyl, or (1-2C)alkanoyl;
A¹, A² and A³ are as defined in relation to Formula (I);
R¹ is a (1-4C)alkyl group;
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, —NR^{a}—CO—, —CONR^{a}—, —S(O)_{z}— (where z is 0, 1 or 2); R^{a} is selected from hydrogen or methyl, and R⁸ is hydrogen or (1-2C)alkyl;
R³ is selected from:
(i) hydrogen, halo, nitro, cyano, or hydroxy;
(ii) an optionally substituted (1-4C)alkyl group wherein the optional substituents are selected from cyano, halo, or a group of sub-formula:

—W—R⁹
wherein W is selected from —O—, —S(O)ₚ— (where p is 0, 1 or 2), —CO—, —NR^{b}CO—, —CONR^{b}—;
R^{b} is selected from hydrogen or (1-2C)alkyl
and R⁹ is selected from hydrogen or (1-2C)alkyl;
   or —NR¹⁰R¹¹, where R¹⁰ and R¹¹ are independently selected from hydrogen or (1-2C)alkyl, or R¹⁰ and R¹¹ are linked to form a 5, or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;
(iii) a group —NR¹²R¹³, wherein R¹² and R¹³ are each independently selected from hydrogen or (1-2C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl; or
(iv) a group of formula (II):

-X-R¹⁴
wherein X is selected from —O—, —S(O)ₚ— (where p is 0, 1 or 2), —CO—, —NR^{c}CO—, or —CONR^{c}—,
R^{c} is selected hydrogen or (1-2C)alkyl, and
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen or (1-2C)alkyl, or R¹⁵ and R¹⁶ are linked to form a 5, or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl; or
(v) a 5-6 membered heterocyclic ring (which may be a heteroaryl ring).

In a further sub-group of compounds of structural Formula IA, or pharmaceutically acceptable salts thereof:

one of A¹, A² or A³ is N, and the others are independently selected from CH or N;
R¹ is a (1-4C)alkyl group;
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, —NR^{a}—CO—, —CONR^{a}—, —S(O)_{z}— (where z is 0, 1 or 2); R^{a} is selected from hydrogen or methyl, and R⁸ is hydrogen or (1-2C)alkyl;
R³ is selected from:
(v) hydrogen, halo, nitro, cyano, or hydroxy;
(vi) an optionally substituted (1-4C)alkyl group wherein the optional substituents are selected from: cyano; halo;
a group of sub-formula:

—W—R⁹
wherein W is selected from —O—, —S(O)ₚ— (where p is 0, 1 or 2), —CO—, —NR^{b}CO—, —CONR^{b}—,
R^{b} is selected from hydrogen or (1-2C)alkyl, and R⁹ is selected from hydrogen or (1-2C)alkyl;
or —NR¹⁰R¹¹, where R¹⁰ and R¹¹ are independently selected from hydrogen or (1-2C)alkyl, or R¹⁰ and R¹¹ are linked to form a 5, or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;
(vii) a group —NR¹²R¹³, wherein R¹² and R¹³ are each independently selected from hydrogen or (1-2C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl; or
(viii) a group of Formula (II):

-X-R¹⁴
wherein X is selected from —O—, —S(O)ₚ— (where p is 0, 1 or 2), —CO—, —NR^{c}CO—, or —CONR^{c}—,
R^{c} is selected hydrogen or (1-2C)alkyl, and
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen or (1-2C)alkyl, or R¹⁵ and R¹⁶ are linked to form a 5, or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl; and
Y is selected from O, S, NR^{y}, or CR^{z}, where R^{y} is selected from hydrogen, (1-2C)alkyl, hydroxy(1-2C)alkyl, (1-2C)alkoxy(1-2C)alkyl, or (1-2C)alkanoyl, and R^{z} is selected from hydrogen, hydroxy, (1-2C)alkyl, hydroxy(1-2C)alkyl, (1-2C)alkoxy(1-2C)alkyl, or (1-2C)alkanoyl; or
(v) a 4-7 membered heterocyclic group which is linked via a carbon atom; and Y is selected from O, S, NR^{y}, or CR^{z}, where R^{y} is selected from hydrogen, (1-2C)alkyl, hydroxy(1-2C)alkyl, (1-2C)alkoxy(1-2C)alkyl, or (1-2C)alkanoyl, and R^{z} is selected from hydrogen, hydroxy, (1-2C)alkyl, hydroxy(1-2C)alkyl, (1-2C)alkoxy(1-2C)alkyl, or (1-2C)alkanoyl.

In a particular group of compounds of Formula IA, Y is selected from O, NR^{y} or CR^{z}, where R^{y} is selected from hydrogen or (1-2C)alkyl, and R^{z} is selected from hydrogen or hydroxy. In a further group of compounds of Formula IA, Y is selected from O or NR^{y}, where R^{y} is selected from hydrogen or (1-2C)alkyl. In a further group of compounds of Formula IA, Y is O.

In compounds of Formula IA, R¹ is suitably has any one of the definitions set out in paragraphs (2) to (7) above. In a particular group of compounds of Formula IA, R¹ is methyl.

In a particular group of compounds of Formula IA, n is as defined in any one of paragraphs (8) to (12) above and R² has any one of the definitions set out herein before or has any one of the definitions set out in paragraphs (13) to (22) above.

In a particular group of compounds of Formula IA, R³ is as defined in either of paragraphs (25) or (26) above.

A further sub-group of compounds of the invention have the structural Formula IB shown below

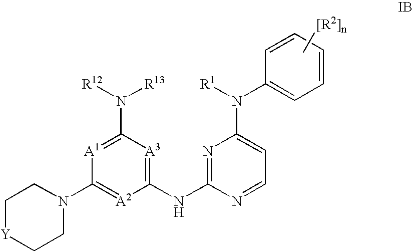

wherein:

A¹, A² and A³ are as defined in relation to Formula (I):

Y, R¹, n and R² each have any one of the definitions set out above in relation to Formula IA;

R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;

or a pharmaceutically acceptable salt thereof.

In a sub-group of compounds of Formula IB, R¹² and R¹³ are suitably linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl.

In a further sub-group of compounds of Formula IB, R¹² and R¹³ are linked to form a 5, 6 or 7-membered heterocyclic ring, and wherein, in addition to the nitrogen atom to which R¹² and R¹³ are attached, the ring optionally comprises one further heteroatom selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl.

A further particular sub-group of compounds of Formula I have the structural Formula IC shown below

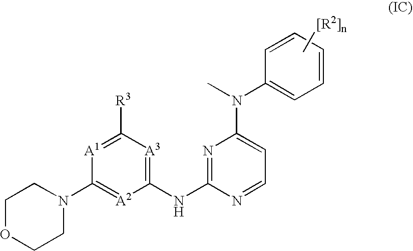

wherein A¹, A² and A³, n, R², and R³ have any one of the definitions set out above in relation to Formula I,
or a pharmaceutically acceptable salt thereof

A further particular sub-group of compounds of the invention are compounds of Formula ID

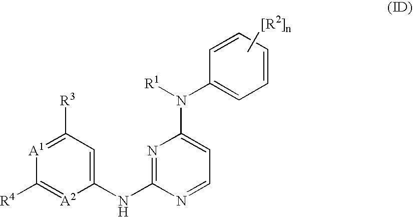

wherein A¹, A², R¹, R², R³, R⁴ and n are as defined in relation to Formula (I). In particular, A¹ is N and A² is CH or N, in particular CH.

A further particular sub-group of compounds of the invention are compounds of Formula IE, or pharmaceutically acceptable salts thereof:

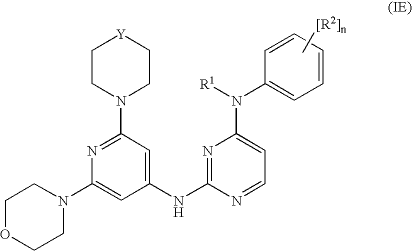

wherein:
R¹ is a (1-4C)alkyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:

-Q-R⁸

where Q is selected from —CO—, —NR^{a}—, —NR^{a}CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R⁸ is hydrogen or (1-2C)alkyl; and
Y is selected from O, —NR^{y}— or —CR^{z}—, where R^{y} is selected from hydrogen or (1-2C)alkyl and R^{z} is selected from hydrogen or hydroxy.

In a sub-group of compounds of Formula IE, Y is selected from O, —NR^{y}— or —CR^{z}—, where R^{y} is methyl and R^{z} is selected from hydrogen or hydroxy.

In a further sub-group of compounds of Formula IE, Y is selected from O or CR^{z}, where R^{z} is selected from hydrogen or hydroxy.

In a further sub-group of compounds of Formula IE, R¹ is (1-4C) alkyl, more particularly, R¹ is methyl.

In a further sub-group of compounds of the Formula (IE), n is 1 or 2, more particularly n is 2.

In a further sub-group of compounds of the Formula (IE), each R² group present is independently selected from (1-2C)alkyl, (1-2C)alkoxy, fluoro, chloro, cyano or hydroxy(1-2C)alkyl, more particularly, each R² group present is independently selected from methyl, methoxy or hydroxymethyl.

Particular novel compounds of the invention include any one of the following:
N′-(3-chloro-2,4-difluoro-phenyl)-N-(4-chloro-6-morpholin-4-yl-pyridin-2-yl)-N′-methyl-pyrimidine-2,4-diamine;
N′-(3-chloro-2,4-difluoro-phenyl)-N-(2,6-dimorpholin-4-ylpyridin-4-yl)-N′-methyl-pyrimidine-2,4-diamine;
N′-(3-chloro-2,4-difluoro-phenyl)-N-(4,6-dimorpholin-4-ylpyridin-2-yl)-N′-methyl-pyrimidine-2,4-diamine;
N′-(3-chloro-2,4-difluoro-phenyl)-N-(2-chloro-6-morpholin-4-yl-pyridin-4-yl)-N′-methyl-pyrimidine-2,4-diamine;
N′-(3-chloro-2,4-difluoro-phenyl)-N-(2,6-dimorpholin-4-ylpyrimidin-4-yl)-N′-methyl-pyrimidine-2,4-diamine;
[3-[[2-[(4-chloro-6-morpholin-4-yl-pyridin-2-yl)amino]pyrimidin-4-yl]-methyl-amino]-4-methyl-phenyl]methanol;
[3-[[2-[(2,6-dimorpholin-4-ylpyridin-4-yl)amino]pyrimidin-4-yl]-methyl-amino]-4-methyl-phenyl]methanol;
3-[[2-[(4,6-dimorpholin-4-ylpyridin-2-yl)amino]pyrimidin-4-yl]-methyl-amino]-4-methyl-phenyl]methanol;
[3-[[2-[(2-chloro-6-morpholin-4-yl-pyridin-4-yl)amino]pyrimidin-4-yl]-methyl-amino]-4-methyl-phenyl]methanol;
[3-[[2-[(2,6-dimorpholin-4-ylpyrimidin-4-yl)amino]pyrimidin-4-yl]-methyl-amino]-4-methyl-phenyl]methanol;

N4-(5-methoxy-2-methylphenyl)-N4-methyl-N2-(2-(4-methylpiperazin-1-yl)-6-morpholinopyridin-4-yl)pyrimidine-2,4-diamine; or
1-(4-(4-((5-methoxy-2-methylphenyl)(methyl)amino)pyrimidin-2-ylamino)-6-morpholinopyridin-2-yl)piperidin-4-ol;
   or a pharmaceutically acceptable salt thereof

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of the invention may be administered in the form of a pro-drug that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I, IA, IB, IC, ID or IE and in vivo cleavable amide derivatives that may be formed at a carboxy group or an amino group in a compound of the Formula I, IA, IB, IC, ID or IE.

Accordingly, the present invention includes those compounds of the Formula I, IA, IB, IC, ID or IE as defined hereinbefore when made available by organic synthesis and when made available within the human or animal body by way of cleavage of a pro-drug thereof. Accordingly, the present invention includes those compounds of the Formula I, IA, IB, IC, ID or IE that are produced by organic synthetic means and also such compounds that are produced in the human or animal body by way of metabolism of a precursor compound, that is a compound of the Formula I, IA, IB, IC, ID or IE may be a synthetically-produced compound or a metabolically-produced compound.

A suitable pharmaceutically-acceptable pro-drug of a compound of the Formula I, IA, IB, IC, ID or IE is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula I, IA, IB, IC or ID that possesses a carboxy group is, for example, an in vivo cleavable ester thereof. An in vivo cleavable ester of a compound of the Formula I containing a carboxy group is, for example, a pharmaceutically-acceptable ester, which is cleaved in the human or animal body to produce the parent acid. Suitable pharmaceutically-acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula I, IA, IB, IC, ID or IE that possesses a hydroxy group is, for example, an in vivo cleavable ester or ether thereof. An in vivo cleavable ester or ether of a compound of the Formula I, IA, IB, IC, ID or IE containing a hydroxy group is, for example, a pharmaceutically-acceptable ester or ether, which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the Formula I, IA, IB, IC, ID or IE that possesses an amino group is, for example, an in vivo cleavable amide derivative thereof. Suitable pharmaceutically-acceptable amides from an amino group include, for example an amide formed with (1-1° C.)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and

4-(1-4C)alkylpiperazin-1-ylmethyl.

The in vivo effects of a compound of the Formula I, IA, IB, IC, ID or IE may be exerted in part by one or more metabolites that are formed within the human or animal body after administration of a compound of the Formula I, IA, IB, IC, ID or IE. As stated hereinbefore, the in vivo effects of a compound of the Formula I, IA, IB, IC, ID or IE may also be exerted by way of metabolism of a precursor compound (a pro-drug).

According to a further aspect of the invention there is provided a pharmaceutical composition, which comprises a compound of the Formula I, IA, IB, IC, ID or IE, or a pharmaceutically acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The compound of Formula I, IA, IB, IC, ID or IE will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the practitioner who is treating any particular patient may determine the optimum dosage.

### Preparation of Compounds of Formula I

It will be appreciated by a person skilled in the art that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Furthermore, the synthesis of optically active forms may be carried out by standard techniques of organic chemistry well known in the art, for example by synthesis from optically active starting materials or by resolution of a racemic form.

Compounds of Formula I can be prepared by various conventional methods as would be apparent to a chemist. In particular, compounds of Formula I may be prepared by reacting a compound of Formula (II):

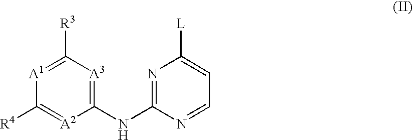

where A¹, A², A³, R³ and R⁴ is as defined in relation to Formula I with the proviso that any functional groups are optionally protected, and L is a leaving group, with a compound of Formula (III)

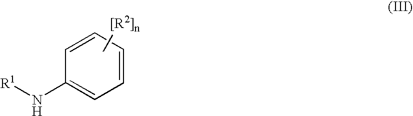

where R¹, n and R² are as defined in relation to Formula I provided that any functional groups are optionally protected. Thereafter, any protecting groups can be removed using conventional methods, and if required, the compound of Formula I can be converted to a different compound of Formula I or a salt, again using conventional chemical methods well known in the art.

Suitable leaving groups L are halogeno such as chloro. The reaction is suitably carried out in an organic solvent such as a C₁₋₆alkanol, for instance, n-butanol, isopropanol or 2-pentanol, dimethylacetamide (DMA), or N-methylpyrrolidine (NMP) or mixtures thereof. An acid, and in particular an inorganic acid such as hydrochloric acid, is suitably added to the reaction mixture. The reaction is suitably conducted at elevated temperatures for example at from 80-150° C., conveniently at the reflux temperature of the solvent.

Alternatively, the reaction between (II) and (III) may be catalysed by transition metals complexes, such as palladium catalysts. Examples of suitable palladium catalysts include Pd2(dba)3 (tris(dibenzylideneacetone)dipalladium), Pd(PPh₃)₄ and Pd(OAc)₂. This palladium catalysed reaction conveniently carried out in the presence of a suitable base, such as potassium carbonate, cesium carbonate, potassium phosphate, sodium tert-butoxide, or 1,8-diazabicyclo[5.4.0]undec-7-ene (DBU). Suitable solvents for such a reaction include toluene, dioxane or ethylene glycol dimethylether (DME). Suitable ligands for use in such a reaction include Xantphos (4,5-bis(diphenylphosphino)-9,9-dimethylxanthene), BINAP (2,2′-bis(diphenylphosphino)-1,1′-binaphtyl) or DPPF (1,1′-bis(diphenylphosphino)ferrocene). The reaction is conveniently carried out at an elevated temperature, generally at the reflux temperature of the particular solvent used. A temperature of 90-140° C. would be typical.

Compounds of Formula (II) may be prepared by various methods including for example, where L is a halogen, by reacting a compound of Formula (IV)

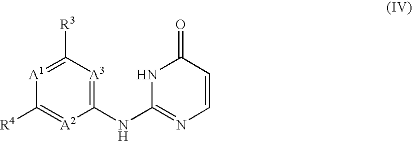

where A¹, A², A³, R³ and R⁴ are as defined in relation to Formula I, with a suitable halogenating agent such as phosphorus oxychloride.

The reaction is conducted under reactions conditions appropriate to the halogenating agent employed. For instance, it may be conducted at elevated temperatures, for example of from 50-100° C., in an organic solvent such as acetonitrile or dichloromethane (DCM).

Compounds of Formula (IV) are suitably prepared by reacting a compound of Formula (V)

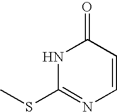

with a compound of Formula (VI)

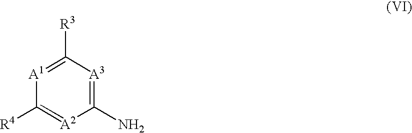

where A¹, A², A³, R³ and R⁴ are as defined in relation to Formula I.

The reaction is suitably effected in an organic solvent such as diglyme, again at elevated temperatures, for example from 120-180° C., and conveniently at the reflux temperature of the solvent.

Compounds of Formula (II), in which L is chloro, may also be prepared by reacting a compound of Formula XIII

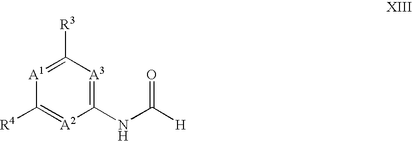

wherein A¹, A², A³, R³ and R⁴ are as defined in relation to Formula I, with 4-chloro-2-methylsulfonylpyrimidine in the presence of a suitable base, such as sodium hydride.

Alternatively, compounds of Formula I may be prepared by reacting a compound of Formula (VII)

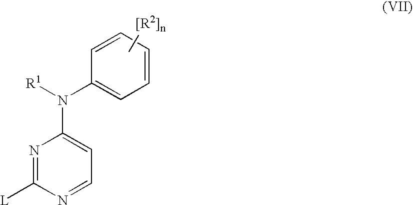

where R¹, n, and R² are as defined in relation to Formula I provided that any functional groups can be optionally protected, and L is a leaving group as defined in relation to Formula (II) or may be —SO₂Me, with a compound of Formula (VI) as defined above.

Again, any protecting groups can be removed using conventional methods, and if required, the compound of Formula I can be converted to a different compound of Formula I or a salt, again using conventional chemical methods.

Conditions for carrying out such a reaction are broadly similar to those required for the reaction between compounds (II) and (III) described above.

Compounds of Formula (VII) are suitably prepared by reacting a compound of Formula (III) as defined above with a compound of Formula (VIII)

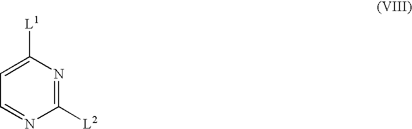

where L¹ and L² are leaving groups such as halogen, and in particular chloro.

The reaction is suitably effected in the presence of an organic base such as triethylamine. The reaction is also suitably carried out at an elevated temperature, for example between 80 and 120° C. in a suitable organic solvent such as a C₁₋₆alkanol, for instance, ethanol. The reaction can also be performed in presence of a strong base such as sodium hydride, in an organic solvent such as DMA. When the basic reaction conditions are used, depressed temperatures, for example from −20° C. to 20° C., conveniently at about 0° C. are suitably employed.

Compounds of Formula (VII) can also be prepared by reacting a compound of Formula (IX)

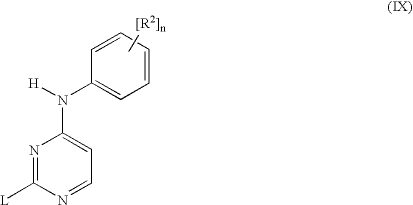

wherein L is a leaving group as defined hereinbefore and R² and n are as defined in relation to Formula I

with a compound

R¹-X

where X is a suitable leaving group such as halogen and R¹ is as defined above in relation to Formula I.

This reaction is conveniently performed using a base such as caesium carbonate in a suitable solvent, such as, for example, dimethylformamide.

Another method to prepare compounds of Formula I involves the reaction of a compound Formula (X)

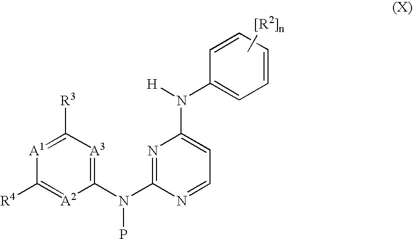

wherein A¹, A², A³, R², n, R³ and R⁴ are as defined above in relation to Formula I; with a compound

R¹-X

where X is a suitable leaving group such as halogen and R¹ is as defined above in relation to Formula I, and P is a suitable protecting group for this reaction, for example a 4-methoxybenzyl group.

This reaction is conveniently performed using a strong base such as sodium hydride in a suitable solvent, for example dimethylformamide.

Another method to prepare compounds of Formula I is to react a compound of Formula (XI)

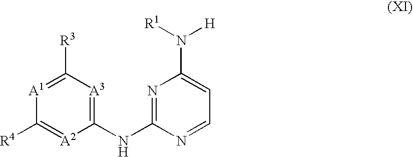

wherein A¹, A², A³, R¹, R³ and R⁴ are as defined above in relation to Formula I; with a compound of Formula (XII)

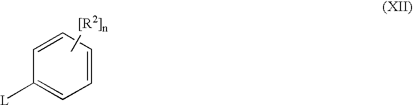

wherein R² and n are as defined above in relation to Formula I and L is halogen, for example bromo.

This reaction is suitably carried out in the presence of a suitable catalyst such as a palladium catalyst. Examples of suitable palladium catalysts include Pd2(dba)3 (tris(dibenzylideneacetone)dipalladium), Pd(PPh₃)₄ and Pd(OAc)₂. This palladium catalysed reaction conveniently carried out in the presence of a suitable base, such as potassium carbonate, cesium carbonate, potassium phosphate, sodium tert-butoxide, or 1,8-diazabicyclo[5.4.0]undec-7-ene (DBU). Suitable solvents for such a reaction include toluene, dioxane or ethylene glycol dimethylether (DME). Suitable ligands for use in such a reaction include Xantphos (4,5-bis(diphenylphosphino)-9,9-dimethylxanthene), BINAP (2,2′-bis(diphenylphosphino)-1,1′-binaphtyl) or DPPF (1,1′-bis(diphenylphosphino)ferrocene). The reaction is conveniently carried out at an elevated temperature, generally at the reflux temperature of the particular solvent used. A temperature of 90-140° C. would be typical.

Compounds of Formula (III) and (VIII) are either known compounds or they can be prepared from known compounds using analogous methods, which would be apparent to the skilled chemist.

Compounds of Formula (VI) are also either known compounds or they can be prepared from known compounds using routine methods.

Compounds of the Formula I can be converted into further compounds of the Formula I using standard procedures conventional in the art. Examples of the types of conversion reactions that may be used to convert a compound of Formula I to a different compound of Formula I include introduction of a substituent by means of an aromatic substitution reaction or of a nucleophilic substitution reaction, reduction of substituents, alkylation of substituents and oxidation of substituents. The reagents and reaction conditions for such procedures are well known in the chemical art.

Particular examples of aromatic substitution reactions include the introduction of an alkyl group using an alkyl halide and Lewis acid (such as aluminium trichloride) under Friedel Crafts conditions; and the introduction of a halogeno group. Particular examples of nucleophilic substitution reactions include the introduction of an alkoxy group or of a monoalkylamino group, a dialkylamino group or a N-containing heterocycle using standard conditions. Particular examples of reduction reactions include the reduction of a carbonyl group to a hydroxy group with sodium borohydride or of a nitro group to an amino group by catalytic hydrogenation with a nickel catalyst or by treatment with iron in the presence of hydrochloric acid with heating.

The preparation of particular compounds of Formula I, such as compounds of Formula IA, IB, IC, ID or IE using the above-described methods form a further aspect of the invention.

### Biological Assays

### A) In Vitro EphB4 Enzyme Assay

This assay detects inhibitors of EphB4-mediated phosphorylation of a polypeptide substrate using Alphascreen™ luminescence detection technology. Briefly, recombinant EphB4 was incubated with a biotinylated-polypeptide substrate (biotin-poly-GAT) in presence of magnesium-ATP. The reaction was stopped by addition of EDTA, together with streptavidin-coated donor beads which bind the biotin-substrate containing any phosphorylated tyrosine residues. Anti-phosphotyrosine antibodies present on acceptor beads bind to phosphorylated substrate, thus bringing the donor & acceptor beads into close proximity. Subsequent excitation of the donor beads at 680 nm generated singlet oxygen species that interact with a chemiluminescer on the acceptor beads, leading to light emission at 520-620 nm. The signal intensity is directly proportional to the level of substrate phosphorylation and thus inhibition is measured by a decrease in signal.

### Aqueous Compound Preparation:

Test compounds were prepared as 10 mM stock solutions in DMSO (Sigma-Aldrich Company Ltd, Gillingham, Dorset SP8 4XT Catalogue No. 154938) and serially diluted with 5% DMSO to give a range of test concentrations at 6× the required final concentration. A 2 μl aliquot of each compound dilution was transferred to appropriate wells of low volume white 384-well assay plates (Greiner, Stroudwater Business Park, Stonehouse, Gloucestershire, GL10 3SX, Cat No. 784075) in duplicate. Each plate also contained control wells: maximum signal was created using wells containing 2 μl of 5% DMSO, and minimum signal corresponding to 100% inhibition were created using wells containing 2 μl of 0.5M EDTA (Sigma-Aldrich Company Ltd, Catalogue No. E7889).

### Acoustic Compound Preparation:

Test compounds were prepared in 100% DMSO and dispensed in multiples of 2.5 nl droplets into the target wells of the assay plate using a Labcyte Echo550 (Sunnyvale, Calif. 94089, USA). To ensure that each well contained a total of 120 nl DMSO the wells were all backfilled as required. Maximum control wells contained DMSO, minimum control wells contained 120 nl of a compound at a concentration sufficient to completely inhibit enzyme activity. The test range of compounds was 100× the required final concentration.

For the assay using aqueous prepared compounds, in addition to the compound or control, each well of the assay plate contained; 10 μl of assay mix containing final buffer (10 mM Tris, 100 μM EGTA, 10 mM magnesium acetate, 4 μM ATP, 500 μM DTT, 1 mg/ml BSA), 0.25 ng of recombinant active EphB4 (amino acids 563-987; Swiss-Prot Acc. No. P54760) (ProQinase GmbH, Breisacher Str. 117, D-79106 Freiburg, Germany, Catalogue No 0178-0000-3) and 5 nM of the poly-GAT substrate (CisBio International, BP 84175, 30204 Bagnols/Cèze Cedex, France, Catalogue No. 61GATBLB). Assay plates were then incubated at room temperature for 1 hour.

For assays using compounds prepared via acoustic dispensing, the assay mix was adjusted such that the final assay volume of 12 ul contained the same concentration of reagent as 10 ul of assay mix used when aqueous compounds were tested.

Regardless of the method of compound preparation, the reaction was stopped by addition of 5 μl/well stop buffer (10 mM Tris, 495 mM EDTA, 1 mg/ml BSA) containing 0.25 ng each of AlphaScreen anti-phosphoTyrosine-100 acceptor beads and streptavidin-coated donor beads (Perkin Elmer, Catalogue No 6760620M). The plates were sealed under natural lighting conditions, wrapped in aluminium foil and incubated in the dark for a further 20 hours.

The resulting assay signal was determined on the Perkin Elmer EnVision plate reader. The minimum value was subtracted from all values, and the signal plotted against compound concentration to generate IC₅₀ data. The method used to generate the compound dilutions was recorded with the IC₅₀ value in the database. Data from compounds prepared using acoustic dispensing were marked “Echo” and the remaining results were marked “Genesis”. Compounds of the invention were tested in the in vitro EphB4 enzyme assay and the IC₅₀ values so obtained are presented in Table A below.

TABLE A

| Example | EphB4 enzyme assay | Method of compound |
|---|---|---|
| Number | IC₅₀ value (μM) | preparation |
| 1 | 38.3 | Genesis |
| 2.1 | 1.12 | Genesis |
| 2.2 | 0.808 | Genesis |
| 2.3 | 1.78 | Genesis |
| 2.4 | 6.27 | Genesis |
| 3 | 0.198 | Genesis |
| 4.1 | 0.297 | Genesis |
| 4.2 | 0.456 | Genesis |
| 4.3 | 0.473 | Genesis |
| 4.4 | 0.374 | Genesis |
| 5.1 | 0.00231 | Echo |
| 5.2 | 0.00325 | Echo |

### B) In Vitro EphB4 Cell Assay

The assay identifies inhibitors of cellular EphB4 by measuring a decrease in phosphorylation of EphB4 following treatment of cells with compound. The endpoint assay used a sandwich ELISA to detect EphB4 phosphorylation status. Briefly, Myc-tagged EphB4 from treated cell lysate was captured on the ELISA plate via an anti-c-Myc antibody. The phosphorylation status of captured EphB4 was then measured using a generic phosphotyrosine antibody conjugated to HRP via a calorimetric output catalysed by HRP, with level of EphB4 phosphorylation directly proportional to the colour intensity. Absorbance was measured spectrophotometrically at 450 nm.

Full length human EphB4 (Swiss-Prot Acc. No. P54760) was cloned using standard techniques from cDNA prepared from HUVEC using RT-PCR. The cDNA fragment was then sub-cloned into a pcDNA3.1 expression vector containing a Myc-His epitope tag to generate full-length EphB4 containing a Myc-His tag at the C-terminus (Invitrogen Ltd. Paisley, UK). CHO-K1 cells (LGC Promochem, Teddington, Middlesex, UK, Catalogue No. CCL-61) were maintained in HAM's F12 medium (Sigma-Aldrich Company Ltd, Gillingham, Dorset SP8 4XT, Catalogue No. N4888) containing 10% heat-inactivated foetal calf serum (PAA lab GmbH, Pasching, Austria Catalogue No. PAA-A15-043) and 1% glutamax-1 (Invitrogen Ltd., Catalogue No. 35050-038) at 37° C. with 5% CO₂. CHO-K1 cells were engineered to stably express the EphB4-Myc-His construct using standard stable transfection techniques, to generate cells hereafter termed EphB4-CHO.

For each assay, 10,000 EphB4-CHO cells were seeded into each well of Costar 96-well tissue-culture plate (Fisher Scientific UK, Loughborough, Leicestershire, UK., Catalogue No. 3598) and cultured overnight in full media. On day 2, the cells were incubated overnight in 90 μl/well of media containing 0.1% Hyclone stripped-serum (Fisher Scientific UK, Catalogue No. SH30068.02). Test compounds were prepared as 10 mM stock solutions in DMSO (Sigma-Aldrich Company Ltd, Gillingham, Dorset SP8 4XT Catalogue No. 154938) and serially diluted with serum-free media to give a range of test concentrations at 10× the required final concentration. A 10 μl aliquot of each compound dilution was transferred to the cell plates in duplicate wells, and the cells incubated for 1 hour at 37° C. Each plate also contained control wells: a maximum signal was created using untreated cells, and minimum signal corresponding to 100% inhibition was created using wells containing a reference compound known to abolish EphB4 activity.

Recombinant ephrin-B2-Fc (R&D Systems, Abingdon Science Park, Abingdon, Oxon OX14 3NB UK, Catalogue No. 496-EB), a Fc-tagged form of the cognate ligand for EphB4, was pre-clustered at a concentration of 3 μg/ml with 0.3 μg/ml anti-human IgG, Fc fragment specific (Jackson ImmunoResearch Labs, Northfield Business Park, Soham, Cambridgeshire, UK CB7 5UE, Catalogue No. 109-005-008) in serum-free media for 30 minutes at 4° C. with occasional mixing. Following compound treatment, cells were stimulated with clustered ephrin-B2 at a final concentration of 1 μg/ml for 20 minutes at 37° C. to induce EphB4 phosphorylation. Following stimulation, the medium was removed and the cells lysed in 100 μl/well of lysis buffer (25 mM Tris HCl, 3 mM EDTA, 3 mM EGTA, 50 mM NaF, 2 mM orthovanadate, 0.27M Sucrose, 10 mM β-glycerophosphate, 5 mM sodium pyrophosphate, 2% Triton X-100, pH 7.4).

Each well of an ELISA Maxisorp 96-well plate (Nunc; Fisher Scientific UK, Loughborough, Leicestershire, UK., Catalogue No. 456537) was coated overnight at 4° C. with 100 μl of anti-c-Myc antibody in Phosphate Buffered Saline (10 μl/ml; produced at AstraZeneca). Plates were washed twice with PBS containing 0.05% Tween-20 and blocked with 250 μl/well 3% TopBlock (Fluka) (Sigma-Aldrich Company Ltd, Gillingham, Dorset SP8 4XT, Catalogue No. 37766) for a minimum of 2 hours at room temperature. Plates were washed twice with PBS/0.05% Tween-20 and incubated with 100 μl/well cell lysate overnight at 4° C. ELISA plates were washed four times with PBS/0.05% Tween-20 and incubated for 1 hour at room temperature with 100 μl/well HRP-conjugated 4G10 anti-phosphotyrosine antibody (Upstate, Dundee Technology Park, Dundee, UK, DD2 1SW, Catalogue No. 16-105) diluted 1:6000 in 3% Top Block. ELISA plates were washed four times with PBS/0.05% Tween-20 and developed with 100 μl/well TMB substrate (Sigma-Aldrich Company Ltd, Catalogue No. T0440). The reaction was stopped after 15 minutes with the addition of 25 μl/well 2M sulphuric acid. The absorbances were determined at 450 nm using the Tecan SpectraFluor Plus. The minimum value was subtracted from all values, and the signal plotted against compound concentration to generate IC₅₀ data.

Compounds of the invention were active in the above assays, for instance, generally showing IC₅₀ values of less than 40 μM in Assay A and 0.4 μM in Assay B. Further illustrative IC₅₀ values obtained using Assay B for a selection of the compounds exemplified in the present application are shown in Table B below.

TABLE B
Mean IC₅₀ values obtained using Assay B

| Example No. | Mean IC₅₀ (μM) |
|---|---|
| 1 | 0.354 |
| 2.1 | 0.008 |
| 3 | 0.028 |
| 4.2 | 0.302 |

As a result of their activity in the screens described above, the compounds of the present invention are expected to be useful in the treatment of diseases or medical conditions mediated alone or in part by EphB4 enzyme activity, i.e. the compounds may be used to produce an EphB4 inhibitory effect in a warm-blooded animal in need of such treatment. Thus, the compounds of the present invention provide a method for treating the proliferation of malignant cells characterised by inhibition of the EphB4 enzyme, i.e. the compounds may be used to produce an anti-proliferative effect mediated alone or in part by the inhibition of EphB4.

According to another aspect of the present invention there is provided a compound of the Formula I, IA, IB, IC, ID or IE or a pharmaceutically acceptable salt thereof, as defined hereinbefore for use in a method of treatment of the human or animal body by therapy.

Thus according to a further aspect of the invention there is provided a compound of the Formula I, IA, IB, IC, ID or IE or a pharmaceutically acceptable salt thereof, as defined hereinbefore for use as a medicament.

According to a further aspect of the invention there is provided the use of a compound of the Formula I, IA, IB, IC, ID or IE, or a pharmaceutically acceptable salt thereof, as defined hereinbefore in the manufacture of a medicament for use in the production of an EphB4 inhibitory effect in a warm-blooded animal such as man.

According to a further feature of this aspect of the invention there is provided a method for producing an EphB4 inhibitory effect in a warm-blooded animal, such as man, in need of such treatment which comprises administering to said animal an effective amount of a compound of the Formula I, IA, IB, IC, ID or IE, or a pharmaceutically acceptable salt thereof, as defined hereinbefore.

According to a further aspect of the invention there is provided the use of a compound of the Formula I, IA, IB, IC, ID or IE, or a pharmaceutically acceptable salt thereof, as defined hereinbefore in the manufacture of a medicament for use in the production of an anti-angiogenic effect in a warm-blooded animal such as man.

According to a further feature of this aspect of the invention there is provided a method for producing an anti-angiogenic effect in a warm-blooded animal, such as man, in need of such treatment which comprises administering to said animal an effective amount of a compound of the Formula I, IA, IB, IC, ID or IE, or a pharmaceutically acceptable salt thereof, as defined hereinbefore.

According to an additional feature of this aspect of the invention there is provided a method of treating cancer in a warm-blooded animal, such as man, in need of such treatment which comprises administering to said animal an effective amount of a compound of the Formula I, IA, IB, IC, ID or IE, or a pharmaceutically acceptable salt thereof, as defined hereinbefore.

According to an additional feature of this aspect of the invention there is provided a compound of the Formula I, IA, IB, IC, ID or IE, or a pharmaceutically acceptable salt thereof, as defined hereinbefore, for use in the treatment of cancer.

According to an additional feature of this aspect of the invention there is provided the use of a compound of the Formula I, IA, IB, IC, ID or IE, or a pharmaceutically acceptable salt thereof, as defined hereinbefore, for use in the manufacture of a medicament for the treatment of cancer.

In a further aspect of the present invention there is provided the use of a compound of the Formula I, IA, IB, IC, ID or IE, or a pharmaceutically acceptable salt thereof, as defined hereinbefore, in the manufacture of a medicament for use in the treatment of solid tumour disease, in particular neuroblastomas, breast, liver, lung and colon cancer or leukemias.

According to an additional feature of this aspect of the invention there is provided a compound of the Formula I, IA, IB, IC, ID or IE or a pharmaceutically acceptable salt thereof, as defined hereinbefore, for use in the treatment of solid tumour disease, in particular neuroblastomas, breast, liver, lung and colon cancer or leukemias.

In a further aspect of the present invention there is provided a method of treating neuroblastomas, breast, liver, lung and colon cancer or leukemias in a warm-blooded animal, such as man, in need of such treatment which comprises administering to said animal an effective amount of a compound of the Formula I, IA, IB, IC, ID or IE or a pharmaceutically acceptable salt thereof, as defined hereinbefore.

The anti-cancer treatment defined hereinbefore may be applied as a sole therapy or may involve, in addition to the compound of the invention, conventional surgery or radiotherapy or chemotherapy. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the anti-angiogenic treatment defined hereinbefore may be: surgery, radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine, taxoids like taxol and taxotere, and polo kinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and bosutinib (SKI-606), and metalloproteinase inhibitors like marimastat and inhibitors of urokinase plasminogen activator receptor function];
(iv) inhibitors of growth factor function: for example such inhibitors include growth factor antibodies and growth factor receptor antibodies [for example the anti-erbB2 antibody trastuzumab and the anti-erbB1 antibodies cetuximab (C225) and panitumumab]; such inhibitors also include, for example, tyrosine kinase inhibitors [for example inhibitors of the epidermal growth factor family (for example EGFR family tyrosine kinase inhibitors such as gefitinib (ZD1839), erlotinib (OSI-774) and CI 1033, and erbB2 tyrosine kinase inhibitors such as lapatinib), inhibitors of the hepatocyte growth factor family, inhibitors of the insulin growth factor receptor, inhibitors of the platelet-derived growth factor family and/or bcr/abl kinase such as imatinib, dasatinib (BMS-354825) and nilotinib (AMN107), inhibitors of cell signalling through MEK, AKT, PI3, c-kit, Flt3, CSF-1R and/or aurora kinases]; such inhibitors also include cyclin dependent kinase inhibitors including CDK2 and CDK4 inhibitors; and such inhibitors also include, for example, inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336);
(v) antiangiogenic agents such as those which inhibit the effects of vascular endothelial growth factor, [for example an anti-vascular endothelial cell growth factor antibody such as bevacizumab (Avastin™) or, for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), or, for example, a compound that works by another mechanism (for example linomide, inhibitors of integrin αᵥβ₃ function and angiostatin)];
(vi) vascular damaging agents such as Combretastatin A4;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination suitable for use in the treatment of cell proliferative disorders (such as solid tumour disease) comprising a compound of Formula I, IA, IB, IC, ID or IE as defined hereinbefore and an additional anti-tumour agent as defined hereinbefore.

According to this aspect of the invention there is provided a pharmaceutical product comprising a compound of Formula I, IA, IB, IC, ID or IE as defined hereinbefore and an additional anti-tumour agent as defined hereinbefore for the conjoint treatment of cancer.

In one embodiment of this aspect of the invention, there is provided a combination suitable for use in the treatment of cell proliferative disorders (such as solid tumour disease) comprising a compound of Formula I, IA, IB, IC, ID or IE as defined hereinbefore, or a pharmaceutically acceptable salt thereof, and a VEGF receptor tyrosine kinase inhibitor, for example 4-(4-fluoro-2-methylindol-5-yloxy)-6-methoxy-7-(3-pyrrolidin-1-ylpropoxy)quinazoline (AZD2171), or a pharmaceutically acceptable salt thereof.

A preferred salt of 4-(4-fluoro-2-methylindol-5-yloxy)-6-methoxy-7-(3-pyrrolidin-1-ylpropoxy)quinazoline (AZD2171) is the maleate salt (AZD2171 maleate) which is described in International Patent Application Publication No. WO 05/061488. AZD2171 maleate salt may be synthesised according to the processes described in WO 05/061488.

In one embodiment of this aspect of the invention, there is provided a pharmaceutical product comprising a compound of Formula I, IA, IB, IC, ID or IE as defined hereinbefore, or a pharmaceutically acceptable salt thereof, and a VEGF receptor tyrosine kinase inhibitor, for example 4-(4-fluoro-2-methylindol-5-yloxy)-6-methoxy-7-(3-pyrrolidin-1-ylpropoxy)quinazoline (AZD2171, or a pharmaceutically acceptable salt thereof.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The combination treatments of the present invention as defined herein are of interest for their antiangiogenic and/or vascular permeability effects. Angiogenesis and/or an increase in vascular permeability is present in a wide range of disease states including cancer (including leukaemia, multiple myeloma and lymphoma), diabetes, psoriasis, rheumatoid arthritis, Kaposi's sarcoma, haemangioma, acute and chronic nephropathies, atheroma, arterial restenosis, autoimmune diseases, acute inflammation, asthma, lymphoedema, endometriosis, dysfunctional uterine bleeding and ocular diseases with retinal vessel proliferation including age-related macular degeneration.

Combination treatments of the present invention are expected to be particularly useful in the prophylaxis and treatment of diseases such as cancer and Kaposi's sarcoma. In particular such combination treatments of the invention are expected to be useful in the treatment of cancer, for example cancer of the lung, head and neck, brain, colon, rectum, oesophagus, stomach, liver, biliary tract, thyroid, kidney, cervix, ovary, uterus, skin, breast, bladder, prostate, pancreas and including haematological malignancies such as leukaemia, multiple myeloma and lymphoma.

In particular such combination treatments of the invention are expected to slow advantageously the growth of primary and recurrent solid tumours.

More particularly such combination treatments of the invention are expected to inhibit any form of cancer associated with VEGF including leukaemia, multiple myeloma and lymphoma and also, for example, to inhibit the growth of those primary and recurrent solid tumours which are associated with VEGF, especially those tumours which are significantly dependent on VEGF for their growth and spread, including for example, certain tumours of the colon, rectum, pancreas, brain, bladder, ovary, breast, prostate, lung, vulva, liver and skin.

In addition to their use in therapeutic medicine, the compounds of Formula I, IA, IB, IC, ID or IE and their pharmaceutically acceptable salts thereof, are also useful as pharmacological tools in the development and standardisation of in vitro and in vivo test systems for the evaluation of the effects of inhibitors of anti-angiogenic activity in laboratory animals such as cats, dogs, rabbits, monkeys, rats and mice, as part of the search for new therapeutic agents.

The invention will now be illustrated in the following Examples in which, generally:

(i) temperatures are given in degrees Celsius (° C.); operations were carried out at room or ambient temperature, that is, at a temperature in the range of 18 to 25° C.;
(ii) organic solutions were dried over anhydrous magnesium sulfate or anhydrous sodium sulfate; evaporation of solvent was carried out using a rotary evaporator under reduced pressure (600 to 4000 Pascals; 4.5 to 30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LC-MS, and reaction times are given for illustration only. The retention times (t_{R}) were measured on a LC/MS Waters 2790/ZMD Micromass system equipped with a Waters Symmetry column (C18, 3.5 μM, 4.6×50 mm) or a Waters Sunfire column (C18, 3.5 μM, 4.6×50 mm); detection UV 254 nM and MS; elution: flow rate 2.5 ml/min, linear gradient from 95% water—5% methanol containing 5% formic acid to 40% water—55% acetonitrile—5% methanol containing 5% formic acid over 3 minutes; then linear gradient to 95% acetonitrile—5% methanol containing 5% formic acid over 1 minute;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 500 MHz using perdeuterio dimethyl sulfoxide (DMSO-d₆) as solvent unless otherwise indicated; the following abbreviations have been used: s, singlet; d, doublet; t, triplet; q, quartet; m, multiplet; br, broad;
(viii) chemical symbols have their usual meanings; SI units and symbols are used;
(ix) solvent ratios are given in volume:volume (v/v) terms; and
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺ which refers to the protonated mass ion; reference to M⁺ is to the mass ion generated by loss of an electron; and reference to M−H⁺ is to the mass ion generated by loss of a proton;
(xi) unless stated otherwise compounds containing an asymmetrically substituted carbon and/or sulfur atom have not been resolved;
(xii) where a synthesis is described as being analogous to that described in a previous example the amounts used are the millimolar ratio equivalents to those used in the previous example;
(xiii) any microwave reactions were carried out in a Personal Chemistry EMRYS™ Optimizer EXP microwave synthesisor;
(xiv) preparative high performance liquid chromatography (HPLC) was performed on a Waters instrument using the following conditions:

| | |
|---|---|
| Column: | 30 mm × 15 cm Xterra Waters, C18, 5 mm |
| Solvent A: | Water with 1% acetic acid or 2 g/l ammonium |
| | carbonate |
| Solvent B: | Acetonitrile |
| Flow rate: | 40 ml/min |
| Run time: | 15 minutes with a 10 minute gradient from 5-95% B |
| Wavelength: | 254 nm |
| Injection volume | 2.0-4.0 ml. |

In addition, the following abbreviations have been used, where necessary:—

NMP 1-methyl-2-pyrrolidinone;

DMA N,N-dimethylacetamide;

DCM Dichloromethane;

DMF N,N-dimethylformamide; and

Ether diethyl ether.

### EXAMPLE 1

### N′-(3-chloro-2,4-difluoro-phenyl)-N-(4-chloro-6-morpholin-4-yl-pyridin-2-yl)-N′-methyl-pyrimidine-2,4-diamine

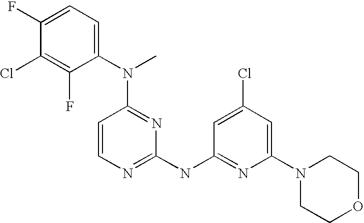

A mixture of 2-chloro-N-(3-chloro-2,4-difluoro-phenyl)-N-methyl-pyrimidin-4-amine (Method 6, 180 mg, 0.62 mmol), 4-chloro-6-morpholin-4-yl-pyridin-2-amine (Method 1, 138 mg, 0.65 mmol), potassium carbonate (1.72 g, 12.5 mmol), Pd2 dba3 (18 mg, 0.031 mmol) and Xantphos (36 mg, 0.062 mmol) in toluene degassed with argon (15 ml) was refluxed for 15 hours. After filtration, the solvent was removed under vacuum and the residue taken up in methylene chloride and purified on silica gel (1 to 4% MeOH in CH₂Cl₂) to give the title compound (230 mg, 79% yield) as a white solid. NMR Spectrum (500 MHz, DMSOd6) 3.35 (s, 3H), 3.37-3.47 (m, 4H), 3.60-3.68 (m, 4H), 6.30 (bs, 1H), 6.37 (s, 1H), 7.26-7.36 (m, 1H), 7.45 (dd, 1H), 7.58 (ddd, 1H), 8.14 (d, 1H), 9.02 (s, 1H). Mass Spectrum: MH⁺ 467.

### EXAMPLE 2

The procedure described in Example 1 was repeated using the appropriate amino heterocycle. Thus were obtained the compounds described below.

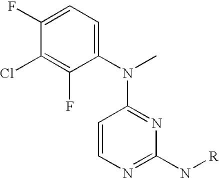

| | | | Molecular | |
|---|---|---|---|---|
| | | R | ion | |
| Example | Name | (starting amine) | (MH⁺) | NMR Spectrum |
| 2.1 | N′-(3-chloro-2,4-difluoro-phenyl)-N-(2,6-dimorpholin-4-ylpyridin-4-yl)-N′-methyl-pyrimidine-2,4-diamine | 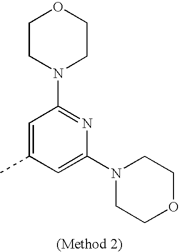 | 518 | 3.24-3.30 (m,8H), 3.38 (s, 3H),3.64-3.69 (m,8H), 5.93 (bs,1H), 6.61 (s, 2H),7.47 (dd, 1H),7.60 (ddd, 1H),8.02 (d, 1H), 9.17(s, 1H) |
| 2.2 | N′-(3-chloro-2,4-difluoro-phenyl)-N-(4,6-dimorpholin-4-ylpyridin-2-yl)-N′-methyl-pyrimidine-2,4-diamine | 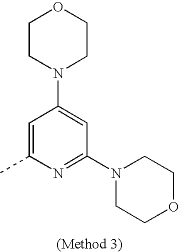 | 518 | 3.10-3.20 (m,4H), 3.35-3.42(m, 7H),3.64-3.72 (m,8H), 5.82 (s, 1H),5.92 (bs, 1H),7.23 (bs, 1H),7.46 (dd, 1H),7.60 (ddd, 1H),8.01 (d, 1H), 8.22(bs, 1H) |
| 2.3 | N′-(3-chloro-2,4-difluoro-phenyl)-N-(2-chloro-6-morpholin-4-yl-pyridin-4-yl)-N′-methyl-pyrimidine-2,4-diamine | 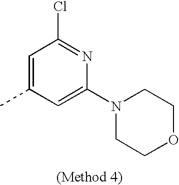 | 467 | 3.25-3.32 (m,4H), 3.37 (s, 3H),3.63-3.69 (m,4H), 6.22 (bs,1H), 7.03 (bs,1H), 7.26-7.35(m, 1H), 7.46 (dd,1H), 7.59 (ddd,1H), 8.13 (s, 1H),8.64 (s, 1H) |
| 2.4 | N′-(3-chloro-2,4-difluoro-phenyl)-N-(2,6-dimorpholin-4-ylpyrimidin-4-yl)-N′-methyl-pyrimidine-2,4-diamine | 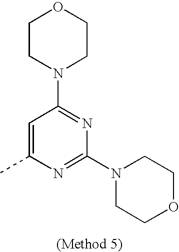 | 519 | 3.34-3.42 (m,7H), 3.53-3.67(m, 12H), 6.03(bs, 1H), 6.93 (bs,1H), 7.47 (dd,1H), 7.60 (ddd,1H), 8.06 (d, 1H),8.66 (s, 1H) |

### EXAMPLE 3

### [3-[[2-[(4-chloro-6-morpholin-4-yl-pyridin-2-yl)amino]pyrimidin-4-yl]-methyl-amino]-4-methyl-phenyl]methanol

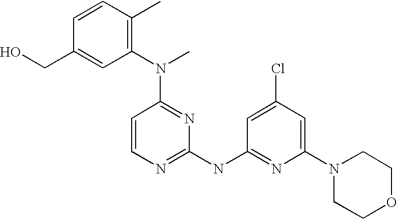

Following the procedure described in Example 1,2-chloro-N-[5-[(dimethyl-tert-butyl-silyl) oxymethyl]-2-methyl-phenyl]-N-methyl-pyrimidin-4-amine (Method 7, 180 mg, 0.48 mmol) and 4-chloro-6-morpholin-4-yl-pyridin-2-amine (Method 1, 107 mg, 0.50 mmol) were reacted to provide N-(4-chloro-6-morpholin-4-yl-pyridin-2-yl)-N′-[5-[(dimethyl -tert-butyl-silyl)oxymethyl]-2-methyl-phenyl]-N′-methyl-pyrimidine-2,4-diamine in 68% yield. NMR Spectrum (500 MHz, CDCl3) 0.09 (s, 6H), 0.92 (s, 9H), 2.13 (s, 3H), 3.44 (s, 3H), 3.48 (bs, 4H), 3.80 (bs, 4H), 4.72 (d, 2H), 5.48 (bs, 1H), 6.24 (s, 1H), 7.12 (s, 1H), 7.24-7.30 (m, 2H), 7.52 (bs, 1H), 7.80 (bs, 1H), 7.95 (bs, 1H). Mass Spectrum: MH⁺ 555. N-(4-chloro-6-morpholin-4-yl-pyridin-2-yl)-N′-[5-[(dimethyl-tert-butyl-silyl)oxymethyl]-2-methyl-phenyl]-N′-methyl-pyrimidine-2,4-diamine (180 mg, 0.32 mmol) was dissolved in methanol (10 ml) and 2N hydrochloric acid was added (0.70 ml). The resulting mixture was stirred at room temperature for 90 minutes and the solvent was evaporated. The residue was neutralized with a minimal amount of ammonium hydroxide and the mixture was extracted with methylene chloride. After evaporation, the residue was triturated in a mixture of diethyl ether and pentane to provide the title compound as a white solid (71 mg, 50% yield). NMR Spectrum (500 MHz, DMSOd6, 353 K) 2.11 (s, 3H), 3.39 (s, 3H), 3.49 (bs, 4H), 3.70 (bs, 4H), 4.53 (d, 2H), 4.96 (bs, 1H), 5.64 (bs, 1H), 6.40 (s, 1H), 7.20 (s, 1H), 7.28 (d, 1H), 7.35 (d, 1H), 7.64 (bs, 1H), 7.93 (d, 1H), 8.41 (bs, 1H). Mass Spectrum: MH⁺ 441.

### EXAMPLE 4

The procedure described in Example 3 was repeated using the appropriate amino heterocycle. Thus were obtained the compounds described below.

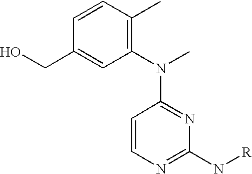

| | | | Molecular | |
|---|---|---|---|---|
| | | R | ion | |
| Example | Name | (starting amine) | (MH⁺) | NMR Spectrum |
| 4.1 | [3-[[2-[(2,6-dimorpholin-4-ylpyridin-4-yl)amino]pyrimidin-4-yl]-methyl-amino]-4-methyl-phenyl]methanol | 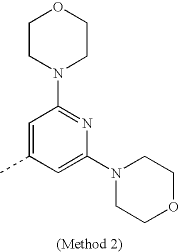 | 492 | 2.08 (s, 3H), 3.32 (bs,8H), 3.36 (s, 3H),3.64-3.71 (m, 8H),4.49 (d, 2H), 5.07 (t,1H), 5.38 (bs, 1H),6.70 (s, 2H), 7.17 (s,1H), 7.25 (d, 1H),7.33 (d, 1H), 7.83 (d,1H), 8.94 (bs, 1H) |
| 4.2 | [3-[[2-[(4,6-dimorpholin-4-ylpyridin-2-yl)amino]pyrimidin-4-yl]-methyl-amino]-4-methyl-phenyl]methanol | 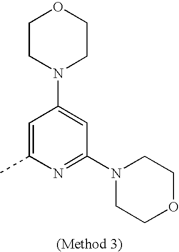 | 492 | 2.08 (s, 3H), 3.25 (bs,3H), 3.34-3.44 (m,8H), 3.63-3.74 (m,8H), 4.49 (d, 2H),5.22 (t, 1H), 5.34 (bs,1H), 5.85 (bs, 1H),7.19 (s, 1H), 7.27 (d,1H), 7.35 (d, 1H),7.45 (bs, 1H), 7.83(bs, 1H), 8.18 (bs, 1H) |
| 4.3 | [3-[[2-[(2-chloro-6-morpholin-4-yl-pyridin-4-yl)amino]pyrimidin-4-yl]-methyl-amino]-4-methyl-phenyl]methanol | 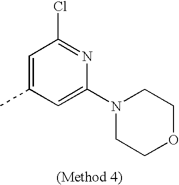 | 441 | 2.08 (s, 3H), 3.37 (bs,7H), 3.64-3.73 (m,4H), 4.50 (s, 2H), 5.08(bs, 1H), 5.44 (bs,1H), 7.17 (s, 1H), 7.24(bs, 2H), 7.26 (d, 1H),7.34 (d, 1H), 7.88 (bs,1H), 9.46 (bs, 1H) |
| 4.4 | [3-[[2-[(2,6-dimorpholin-4-ylpyrimidin-4-yl)amino]pyrimidin-4-yl]-methyl-amino]-4-methyl-phenyl]methanol | 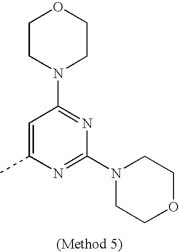 | 493 | 2.11 (s, 3H), 3.39 (s,3H), 3.50 (bs, 4H),4.59-4.76 (m, 12H),4.53 (s, 2H), 5.13 (s,1H), 5.47 (bs, 1H),7.20 (bs, 1H), 7.21 (s,1H), 7.30 (d, 1H),7.37 (d, 1H), 7.91 (bs,1H), 8.34 (bs, 1H) |

### EXAMPLE 5

The procedure described in Example 1 was repeated using the appropriate amino heterocycle. Thus were obtained the compounds described below.

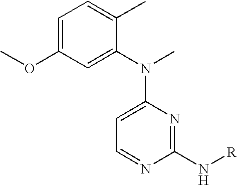

| Example | Name | R (starting amine) | MH⁺ | NMR Spectrum |
|---|---|---|---|---|
| 5.1^{a} | N4-(5-methoxy-2-methylphenyl)-N4-methyl-N2-(2-(4-methylpiperazin-1-yl)-6-morpholinopyridin-4-yl)pyrimidine-2,4-diamine | 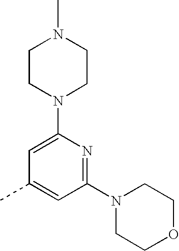 | 505 | 2.06 (s, 3H), 2.85 (s,3H), 3.04-3.18 (m, 4H),3.38-3.44 (m, 4H), 3.48(s, 3H), 3.49-3.54 (m,2H), 3.66-3.72 (m, 4H),3.75 (s, 3H), 4.25-4.32(m, 2H), 5.67 (d, 1H),6.50 (s, 1H), 6.56 (s,1H), 6.93 (d, 1H), 7.00(dd, 1H), 7.33 (d, 1H),7.85 (d, 1H) |
| 5.2^{b} | 1-(4-(4-((5-methoxy-2-methylphenyl)(methyl)amino)pyrimidin-2-ylamino)-6-morpholinopyridin-2-yl)piperidin-4-ol | 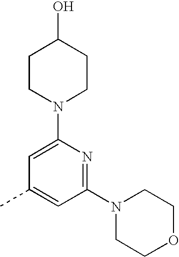 | 506 | 1.28-1.42 (m, 2H), 1.70-1.82 (m, 2H), 2.01 (s,3H), 2.87-3.02 (m, 2H),3.27-3.34 (m, 4H), 3.38(s, 3H), 3.64 (dddd, 1H),3.58-3.71 (m, 5H), 3.74(s, 3H), 3.92 (bs, 2H),4.65 (d, 1H), 5.36 (bs,1H), 6.64 (bs, 1H), 6.81(bs, 1H), 6.87 (d, 1H),6.92 (d, 1H), 7.29 (d,1H), 7.83 (bs, 1H), 9.08(bs, 1H) |

^{a}2-chloro-N-(5-methoxy-2-methylphenyl)-N-methylpyrimidin-4-amine (190 mg, 0.72 mmol, method 10), 2-(4-methylpiperazin-1-yl)-6-morpholinopyridin-4-amine (210 mg, 0.76 mmol, method 8) were reacted as described in Example 1. Purification was done by HPLC using a Waters X-Bridge reverse-phase column (5 microns silica, 19 mm diameter, 100 mm length) and decreasingly polar mixtures of water (containing 0.2% ammonium carbonate) and acetonitrile as eluent. The fractions were evaporated to dryness to afford the desired product (65.0 mg, 17.88%) as a pale yellow foam.
^{b}2-chloro-N-(5-methoxy-2-methylphenyl)-N-methylpyrimidin-4-amine (159 mg, 0.60 mmol, method 10), 1-(4-amino-6-morpholinopyridin-2-yl)piperidin-4-ol (140 mg, 0.50 mmol, method 9), were reacted as described in Example 1 except that after 4 hrs, more 2-chloro-N-(5-methoxy-2-methylphenyl)-N-methylpyrimidin-4-amine (66.3 mg, 0.25 mmol), tris(dibenzylideneacetone)dipalladium (23.03 mg, 0.03 mmol) and 9,9-dimethyl-4,5-bis(diphenylphosphino)xanthene (29.1 mg, 0.05 mmol) were added and the reaction mixture was refluxed for an additional 4 hrs. Reaction mixture was eluted through a SCX-2 column (10 g) with 1) dichloromethane, 2) methanol and 3) methanolic ammonia 7N. Desired fractions were collected and concentrated to dryness to afford a dark brown residue. The crude product was purified by flash chromatography on silica gel eluting with 0 to 100% ethyl acetate in dichloromethane. The solvent was evaporated to dryness and the resulting pale pink foam was further purified by preparative HPLC using a Waters X-Terra reverse-phase column (C-18, 5 microns silica, 19 mm diameter, 100 mm length, flow rate of 40 ml/minute) using decreasingly polar mixtures of water (containing 0.2% ammonium carbonate) and acetonitrile as eluent to afford 1-(4-(4-((5-methoxy-2-methylphenyl)(methyl)amino)pyrimidin-2-ylamino)-6-morpholinopyridin-2-yl)piperidin-4-ol (40.0 mg, 15.73%) as a pale yellow foam.

### Method 1

### 4-chloro-6-morpholin-4-yl-pyridin-2-amine

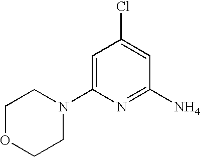

A mixture of 2-amino-4,6-dichloro-pyridine (900 mg, 5.52 mmol, described in Rec. Trav. Chim. Pays-Bas 1950, vol. 69, p. 673-690), morpholine (4.80 ml, 55.2 mmol) and DMSO (0.39 ml, 5.52 mmol) was heated on a Personal Chemistry EMRYS™ Optimizer EXP microwave synthesisor at 170° C. for 30 minutes. After cooling, morpholine was removed in vacuo, the residue was treated with 30% ammonium hydroxide and the resulting mixture was extracted with methylene chloride. Evaporation of the solvent and purification of the residue on silica gel (3 to 4% MeOH in CH₂Cl₂) provided 4-chloro-6-morpholin-4-yl-pyridin-2-amine (890 mg, 72% yield) and its isomer 6-chloro-4-morpholin-4-yl-pyridin-2-amine (190 mg, 16% yield). NMR Spectrum (500 MHz, CDCl3) 3.43-3.45 (m, 4H), 3.76-3.78 (m, 4H), 4.30 (bs, 2H), 5.91 (s, 1H), 5.97 (s, 1H). Mass Spectrum: MH⁺ 214.

### Method 2

### 2,6-dimorpholin-4-ylpyridin-4-amine

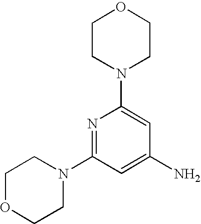

A mixture of 4-amino-2,6-dichloro-pyridine (900 mg, 5.52 mmol), morpholine (4.8 ml, 55.2 mmol) and DMA (1.0 ml, 11 mmol) was heated on a Personal Chemistry EMRYS™ Optimizer EXP microwave synthesisor at 240° C. for 30 minutes. After cooling, morpholine was removed in vacuo, the residue was treated with 30% ammonium hydroxide and the resulting mixture was extracted with methylene chloride. Evaporation of the solvent and purification of the residue on silica gel (3% MeOH in CH₂Cl₂) followed by trituration in ether provided 2,6-dimorpholin-4-ylpyridin-4-amine (950 mg, 65% yield). NMR Spectrum (500 MHz, CDCl3) 3.39-3.42 (m, 8H), 3.77-3.79 (m, 8H), 3.91 (bs, 2H), 5.39 (s, 2H). Mass Spectrum: MH⁺ 265.

### Method 3

### 4,6-dimorpholin-4-ylpyridin-2-amine

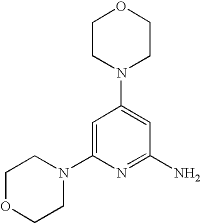

A mixture of 2-amino-4,6-dichloro-pyridine (2.0 g, 12.2 mmol), morpholine (10.7 ml, 123 mmol) and DMSO (0.87 ml, 12 mmol) was heated on a Personal Chemistry EMRYS™ Optimizer EXP microwave synthesisor at 220° C. for 20 minutes. After cooling, morpholine was removed in vacuo, the residue was treated with 30% ammonium hydroxide and the resulting mixture was extracted with methylene chloride. Evaporation of the solvent and purification of the residue on silica gel (3 to 4% MeOH in CH₂Cl₂) provided a gum that was triturated in ether to give 4,6-dimorpholin-4-ylpyridin-2-amine (900 mg, 28% yield). NMR Spectrum (500 MHz, CDCl3) 3.20-3.22 (m, 4H), 3.40-3.42 (m, 4H), 3.79-3.81 (m, 8H), 4.20 (bs, 2H), 5.44 (s, 1H), 5.46 (s, 1H). Mass Spectrum: MH⁺ 265.

### Method 4

### 2-chloro-6-morpholin-4-yl-pyridin-4-amine

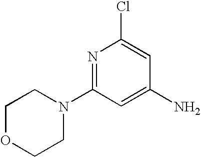

A mixture of 4-amino-2,6-dichloro-pyridine (2.0 g, 12.2 mmol), morpholine (10.7 ml, 123 mmol) and DMSO (0.87 ml, 12.3 mmol) was heated on a Personal Chemistry EMRYS™ Optimizer EXP microwave synthesisor at 170° C. for 1 hours. After cooling, morpholine was removed in vacuo, the residue was treated with 30% ammonium hydroxide and the resulting mixture was extracted with methylene chloride. Evaporation of the solvent and purification of the residue on silica gel (2 to 3% MeOH in CH₂Cl₂) provided 2-chloro-6-morpholin-4-yl-pyridin-4-amine (1.4 g, 53% yield). NMR Spectrum (500 MHz, CDCl3) 3.42-3.44 (m, 4H), 3.76-3.78 (m, 4H), 4.07 (bs, 2H), 5.69 (s, 1H), 6.04 (s, 1H). Mass Spectrum: MH⁺ 214.

### Method 5

### 2,6-dimorpholin-4-ylpyrimidin-4-amine

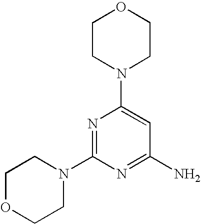

A mixture of 4-amino-2,6-dichloropyrimidine (1.64 g, 10.0 mmol), morpholine (8.7 ml, 100 mmol) and DMA (1.90 ml, 20 mmol) was heated at 180° C. for 1 hour. After cooling, morpholine was removed in vacuo and the residue was purified on silica gel (2 to 4% MeOH in CH₂Cl₂) to provide 2,6-dimorpholin-4-ylpyrimidin-4-amine (2.1 g, 79% yield) as a white solid. NMR Spectrum (500 MHz, CDCl3) 3.45-3.49 (m, 4H), 3.71-3.75 (m, 12H), 4.37 (bs, 2H), 5.07 (s, 1H). Mass Spectrum: MH⁺ 266.

### Method 6

### 2-chloro-N-(3-chloro-2,4-difluoro-phenyl)pyrimidin-4-amine

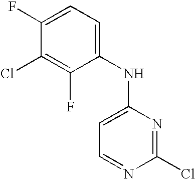

A mixture of 2,4-dichloropyrimidine (6.0 g, 40.5 mmol), 3-chloro-2,4-difluoroaniline (6.28 g, 38.5 mmol), diisopropylethylamine (9.16 ml, 52.7 mmol) and pentanol (20 ml) was refluxed for 24 hours. After concentration under reduced pressure, the residue was dissolved in ethyl acetate and the solution washed with water, dried and concentrated. The crude material was triturated in methylene chloride and the white solid collected by filtration to give 2.1 g of the desired product. The filtrate was concentrated and purified on silica gel (10 to 50% EtOAc in petroleum ether) to give another 1.9 g of material (total yield 4.0 g, 36%). NMR Spectrum (500 MHz, DMSOd6) 6.80 (d, 1H), 7.38 (ddd, 1H), 7.74 (ddd, 1H), 8.21 (d, 1H), 9.95 (bs, 1H); Mass Spectrum MH⁺ 276.

2-chloro-N-(3-chloro-2,4-difluoro-phenyl)-N-methyl-pyrimidin-4-amine

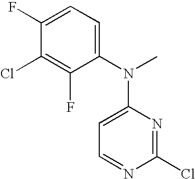

Iodomethane (0.55 mL, 8.91 mmol) was added dropwise to a suspension of 2-chloro-N-(3-chloro-2,4-difluoro-phenyl)pyrimidin-4-amine (2.45 g, 8.91 mmol) and Cs₂CO₃ (5.79 g, 17.8 mmol) in 15 mL acetonitrile and the mixture was stirred overnight. After evaporation under reduced pressure, the residue was dissolved in methylene chloride and the solution was filtered and evaporated. Purification of the residue on silica gel (10 to 30% EtOAc in petroleum ether) gave 2-chloro-N-(3-chloro-2,4-difluoro-phenyl)-N-methyl-pyrimidin-4-amine (2.20 g, 86%) as an oil which solidified on standing. NMR Spectrum (500 MHz, DMSO-d6) 3.36 (s, 3H), 6.53 (bs, 1H), 7.49 (ddd, 1H), 7.62 (ddd, 1H), 8.15 (bs, 1H); Mass Spectrum MH⁺ 290.

### Method 7

### [3-[(2-chloropyrimidin-4-yl)amino]-4-methyl-phenyl]methanol

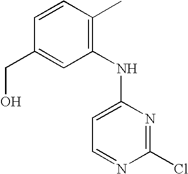

A mixture of 2,4-dichloropyrimidine (4 g, 27 mmol), (3-amino-4-methylphenyl)methanol (3.7 g, 27 mmol), triethylamine (4.13 ml, 29.7 mmol) in ethanol (20 ml) was refluxed for 18 hours. After concentration under reduced pressure, the residue was dissolved in ethyl acetate and the solution was washed with water, dried and concentrated. The crude material was purified by chromatography on silica gel (eluant: 5% methanol in DCM). After collection of the fractions and evaporation of the solvent, the residue was stirred in DCM (60 ml), filtered and dried under vacuum to give the title compound (2.6 g, 39%). NMR Spectrum (500 MHz, DMSO-d6) 2.15 (s, 3H), 4.46 (d, 2H), 5.18 (t, 1H), 6.44 (m, 1H), 7.14 (d, 1H), 7.22 (s, 1H), 7.25 (d, 1H), 8.07 (d, 1H), 9.55 (bs, 1H); Mass Spectrum MH⁺ 250.

### 2-chloro-N-[5-[(dimethyl-tert-butyl-silyl)oxymethyl]-2-methyl-phenyl]pyrimidin-4-amine

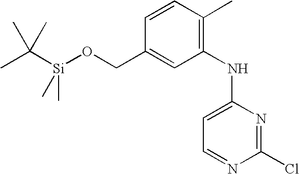

A mixture of [3-[(2-chloropyrimidin-4-yl)amino]-4-methyl-phenyl]methanol (20.0 g, 80.1 mmol), tert-butyldimethylsilyl chloride (18.1 g, 120 mmol) and imidazole (16.3 g, 240 mmol) in DMF (150 ml) was stirred at room temperature for 16 hours. After evaporation of the solvent under reduced pressure, the residue was taken in diethyl ether and washed with water. The organic phase was concentrated and purified on silica gel (10% EtOAc in CH₂Cl₂) to provide the title compound as a colorless oil (19.3 g, 66% yield). NMR Spectrum (500 MHz, DMSO-d6) 0.11 (s, 6H), 0.93 (s, 9H), 2.20 (s, 3H), 4.72 (s, 2H), 6.52 (bs, 1H), 7.15 (d, 1H), 7.29-7.31 (m, 2H), 8.11 (d, 1H), 9.57 (bs, 1H). Mass Spectrum MH⁺ 364.

### 2-chloro-N-[5-[(dimethyl-tert-butyl-silyl)oxymethyl]-2-methyl-phenyl]-N-methyl-pyrimidin-4-amine

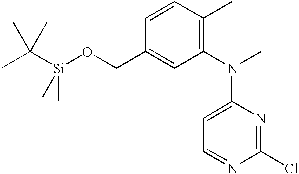

Iodomethane (0.85 ml, 13.7 mmol) was added dropwise to a suspension of 2-chloro-N-[5-[(dimethyl-tert-butyl-silyl)oxymethyl]-2-methyl-phenyl]pyrimidin-4-amine (5.0 g, 13.7 mmol) and Cs2CO3 (8.94 g, 27.5 mmol) in acetonitrile (30 ml) at room temperature. The mixture was stirred at 45° C. for 3 hours. After evaporation under reduced pressure, the residue was dissolved in DCM and the solution was filtered and evaporated.

Purification of the residue by chromatography on silica gel (0 to 10% EtOAc in CH₂Cl₂) gave

### 2-chloro-N-[5-[(dimethyl-tert-butyl-silyl)oxymethyl]-2-methyl-phenyl]-N-methyl-pyrimidin-4-amine (5.2 g, 100%) as an oil. NMR Spectrum (500 MHz, DMSOd6) 0.10 (s, 6H), 0.92 (s, 9H), 2.10 (s, 3H), 3.36 (s, 3H), 4.74 (s, 2H), 5.83 (d, 1H), 7.23 (s, 1H), 7.35 (d, 1H), 7.44 (d, 1H), 7.97 (d, 1H). Mass Spectrum MH⁺ 378.

Method 8

### 2-(4-methylpiperazin-1-yl)-6-morpholinopyridin-4-amine

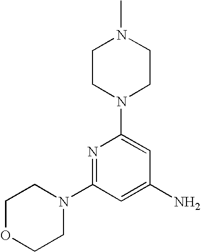

2-chloro-6-morpholinopyridin-4-amine (300 mg, 1.40 mmol, from method 4), n-methylpiperazine (3.11 mL, 28.08 mmol) were dissolved in DMA (2 mL) and heated in an autoclave at 190° C. for 20 hours. The reaction mixture was concentrated to dryness, diluted with dichloromethane, washed with a saturated aqueous solution of sodium hydrogencarbonate. The aqueous layer was extracted with dichloromethane. The organic phases were combined, washed with a saturated aqueous solution of brine, dried over magnesium sulfate and concentrated to afford the crude product as a dark orange oil. The reaction mixture was purified by preparative HPLC using a Waters X-Bridge reverse-phase column (5 microns silica, 19 mm diameter, 100 mm length) and decreasingly polar mixtures of water (containing 0.2% ammonium carbonate) and acetonitrile as eluent. The fractions were evaporated to dryness to afford 2-(4-methylpiperazin-1-yl)-6-morpholinopyridin-4-amine (225 mg, 57.8%) as a yellow foam. NMR Spectrum (500 MHz, CDCl3): 2.33 (s, 3H), 2.46-2.53 (m, 4H), 3.38-3.43 (m, 4H), 3.44-3.50 (m, 4H), 3.76-3.81 (m, 4H), 3.86 (bs, 2H), 5.37 (d, 1H), 5.42 (d, 1H)

### Method 9

### 1-(4-amino-6-morpholinopyridin-2-yl)piperidin-4-ol

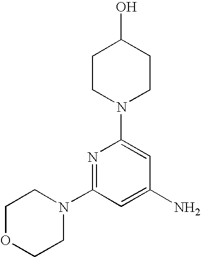

2-chloro-6-morpholinopyridin-4-amine (0.40 g, 1.87 mmol, from method 4) and 4-hydroxypiperidine (2.84 g, 28.08 mmol) were dissolved in DMA (3 mL) and sealed into a microwave tube. The reaction was heated to 25° C. over a period of 1 hour and 10 minutes in a microwave reactor. The reaction mixture was quenched with water, saturated with sodium chloride and extracted with ethyl acetate. The combined organic phases were washed with brine, dried over magnesium sulfate and concentrated to afford the crude product as a dark orange oil. The reaction mixture was purified by preparative HPLC using a Waters X-Terra reverse-phase column (C-18, 5 microns silica, 19 mm diameter, 100 mm length, flow rate of 40 ml/minute) and decreasingly polar mixtures of water (containing 0.2% ammonium carbonate) and acetonitrile as eluent. The fractions containing the desired compound were evaporated to dryness to afford 1-(4-amino-6-morpholinopyridin-2-yl)piperidin-4-ol (160 mg, 30.7%) as a yellow crystalline solid. Mass Spectrum: M+H⁺279. NMR Spectrum (500 MHz, DMSOd6+TFAd): 1.45-1.57 (m, 2H), 1.80-1.90 (m, 2H), 3.03-3.13 (m, 2H), 3.19-3.27 (m, 4H), 3.49-3.59 (m, 2H). 3.70-3.78 (m, 5H), 5.56 (s, 1H), 5.64 (s, 1H)

### Method 10

### 2-chloro-N-(5-methoxy-2-methyl-phenyl)pyrimidin-4-amine

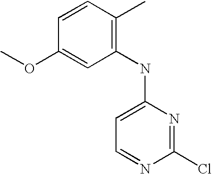

A mixture of 2,4-dichloropyrimidine (6 g, 40 mmol), 5-methoxy-2-methylaniline (5.5 g, 40 mmol), triethylamine (6.2 ml, 44.6 mmol) in ethanol (60 ml) was refluxed for 18 hrs. After concentration under reduced pressure, the residue was dissolved in ethyl acetate and the solution was washed with water, dried and concentrated. The crude product was purified on silica gel (10 to 40% ethyl acetate in petroleum ether) to provide the title compound in 40% yield. NMR Spectrum (500 MHz, DMSOd6) 2.11 (s, 3H), 3.72 (s, 3H), 6.52 (bs, 1H), 6.78 (dd, 1H), 6.95 (s, 1H), 7.19 (d, 1H), 8.08 (d, 1H), 9.49 (bs, 1H).

### 2-chloro-N-(5-methoxy-2-methyl-phenyl)-N-methyl-pyrimidin-4-amine

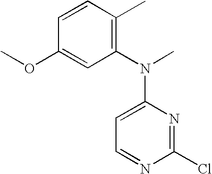

Iodomethane (1.1 ml, 17.7 mmol) was added dropwise to a suspension of 2-chloro-N-(5-methoxy-2-methyl-phenyl)pyrimidin-4-amine (4.0 g, 16.1 mmol) and Cs₂CO₃ (10.4 g, 32.1 mmol) in DMF (25 ml) at room temperature. The mixture was stirred at room temperature overnight. After evaporation under reduced pressure, the residue was dissolved in DCM and the solution was filtered and evaporated. Purification of the residue by chromatography on silica gel (10 to 40% EtOAc in petroleum ether) gave the title compound (3.7 g, 88%) as a viscous oil. NMR Spectrum (500 MHz, DMSO-d6) 2.00 (s, 3H), 3.30 (s, 3H), 3.74 (s, 3H), 5.84 (d, 1H), 6.91 (s, 1H), 6.95 (d, 1H), 7.33 (d, 1H), 7.93 (d, 1H); Mass Spectrum MH⁺ 264.

1. A compound of Formula I 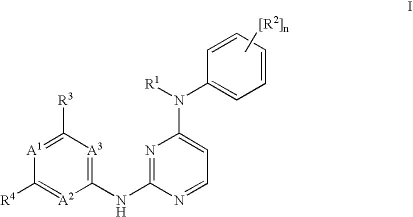
wherein:
one of A¹, A² or A³ is N, and the others are independently selected from CH or N;
R¹ is a (1-4C)alkyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
n is 0, 1, 2 or 3;
each R² group present is independently selected from (1-2C)alkyl, (1-2C)alkoxy, fluoro, chloro, cyano, hydroxy(1-2C)alkyl, or a group of sub-formula:
-Q-R⁸
where Q is selected from —CO—, —NR^{a}—, —NR^{a}—CO—, —NR^{a}−COO—, NR^{a}CONR^{b}, —CONR^{a}—, —S(O)_{z}— (where z is 0, 1 or 2); —SO₂NR^{a}—, and —NR^{a}SO₂, R^{a} and R^{b} are each independently selected from hydrogen or methyl, and R⁸ is hydrogen or (1-2C)alkyl;
R³ is selected from:
(i) hydrogen, halo, nitro, cyano, or hydroxy;
(ii) an optionally substituted (1-6C)alkyl, (2-6C)alkenyl, or (2-6C)alkynyl group wherein the optional substituents are selected from: cyano; halo; a group of sub-formula:
—W—R⁹
wherein W is selected from —O—, —S(O)ₚ— (where p is 0, 1 or 2), —CO—, —NR^{b}CO—, —CONR^{b}—, —NR^{b}CONR^{b}—, —SO₂NR^{b}—, —NR^{b}SO₂—, or —NR^{b}COO—;
R^{b} is selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen, or (1-2C)alkyl, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iii) a group —NR¹²R¹³, wherein R¹² and R¹³ are each independently selected from hydrogen or (1-6C)alkyl, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iv) a group of formula (II):
-X-R¹⁴
wherein X is selected from —O—, —S(O)ₚ— (where p is 0, 1 or 2), —CO—, —NR^{c}CO—, —CONR^{c}—, —NR^{c}COO—, and —NR^{c}SO₂—,
where R^{c} is selected hydrogen or (1-2C)alkyl;
R¹⁴ is a (1-4C)alkyl group which is optionally substituted by halo, hydroxy, cyano, (1-4C)alkoxy, or R¹⁴ is
—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl; or
(v) a 4-7 membered heterocyclic group which is linked via a carbon atom;
R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
or a pharmaceutically acceptable salt thereof.

2. The compound according to claim 1, or a pharmaceutically acceptable salt thereof, wherein:
A¹ is N and A² and A³ are CH;
A² is N and A¹ and A³ are CH; or
A¹ and A² are N and A³ is CH.

3. The compound according to claim 2, or a pharmaceutically acceptable salt thereof, wherein A¹ is N and A² and A³ are CH.

4. The compound according to claim 1 an on of claim 1, or a pharmaceutically acceptable salt thereof, wherein R¹ represents (1-4C)alkyl.

5. The compound according to claim 1, or a pharmaceutically acceptable salt thereof, wherein n is 2 or 3 and each R² group present is independently selected from methyl, fluoro, chloro, hydroxymethyl or methoxy.

6. The compound according to claim 1, or a pharmaceutically acceptable salt thereof, wherein R³ is selected from a nitrogen linked piperidinyl, piperazinyl or morpholinyl ring and wherein any carbon atom present in the ring is optionally substituted by hydroxy and the available nitrogen atom present in the piperazinyl ring is optionally substituted by methyl.

7. The compound according claim 1, or a pharmaceutically acceptable salt thereof, wherein R⁴ represents morpholinyl.

8. The compound according to claim 1 selected from:
N′-(3-chloro-2,4-difluoro-phenyl)-N-(4-chloro-6-morpholin-4-yl-pyridin-2-yl)-N′-methyl-pyrimidine-2,4-diamine;
N′-(3-chloro-2,4-difluoro-phenyl)-N-(2,6-dimorpholin-4-yl-pyridin-4-yl)-N′-methyl-pyrimidine-2,4-diamine;
N′-(3-chloro-2,4-difluoro-phenyl)-N-(4,6-dimorpholin-4-yl-pyridin-2-yl)-N′-methyl-pyrimidine-2,4-diamine;
N′-(3-chloro-2,4-difluoro-phenyl)-N-(2-chloro-6-morpholin-4-yl-pyridin-4-yl)-N′-methyl-pyrimidine-2,4-diamine;
N′-(3-chloro-2,4-difluoro-phenyl)-N-(2,6-dimorpholin-4-ylpyrimidin-4-yl)-N′-methyl-pyrimidine-2,4-diamine;
[3-[[2-[(4-chloro-6-morpholin-4-yl-pyridin-2-yl)amino]pyrimidin-4-yl]-methyl-amino]-4-methyl-phenyl]methanol;
[3-[[2-[(2,6-dimorpholin-4-ylpyridin-4-yl)amino]pyrimidin-4-yl]-methyl-amino]-4-methyl-phenyl]methanol;
3-[[2-[(4,6-dimorpholin-4-ylpyridin-2-yl)amino]pyrimidin-4-yl]-methyl-amino]-4-methyl-phenyl]methanol;
[3-[[2-[(2-chloro-6-morpholin-4-yl-pyridin-4-yl)amino]pyrimidin-4-yl]-methyl-amino]-4-methyl-phenyl]methanol;
[3-[[2-[(2,6-dimorpholin-4-ylpyrimidin-4-yl)amino]pyrimidin-4-yl]-methyl-amino]-4-methyl-phenyl]methanol;
N4-(5-methoxy-2-methylphenyl)-N4-methyl-N2-(2-(4-methylpiperazin-1-yl)-6-morpholinopyridin-4-yl)pyrimidine-2,4-diamine; and
1-(4-(4-((5-methoxy-2-methylphenyl)(methyl)amino)pyrimidin-2-ylamino)-6-morpholinopyridin-2-yl)piperidin-4-ol
and pharmaceutically acceptable salts thereof.

9. A pharmaceutical composition which comprises a compound according to any one of claims 1 to 8, or a pharmaceutically acceptable salt thereof, in association with a pharmaceutically acceptable diluent or carrier.

10-12. (canceled)

13. A process for the manufacture of a compound of Formula I as defined in claim 1 which comprises reacting a compound of Formula (VII), 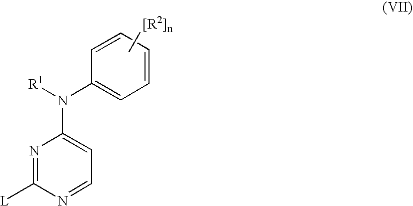
wherein L is halogen and any functional groups are optionally protected, with a compound of Formula (VI), 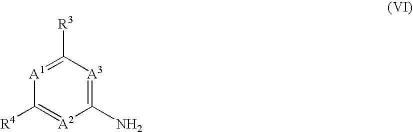
and thereafter, optionally:
(i) converting a compound of Formula (I) into another compound of Formula (I);
(ii) removing any protecting groups; and/or
(iii) forming a salt thereof.

14. A method for producing an anti-angiogenic effect in a warm-blooded animal in need thereof, which comprises administering to said animal an effective amount of a compound of the Formula (I) according to claim 1.

15. A method for the treatment of a solid tumour disease in a warm-blooded animal in need thereof, which comprises administering to said animal an effective amount of a compound of the Formula (I) according to claim 1.

16. The method of claim 15 wherein said solid tumour disease is selected from neuroblastomas, breast, liver, lung and colon cancer and leukemias.
